(19) United States
(12) Patent Application Publication
(10) Pub. No.: US 2014/0291551 A1
(43) Pub. Date: Oct. 2, 2014

(54) METHODS AND SYSTEMS FOR DETECTING AND QUANTIFYING PETROLEUM OIL BASED ON FLUORESCENCE

(71) Applicant: AXURE TECHNOLOGIES S.A., Bogota (CO)

(72) Inventors: Raul Cuero Rengifo, Cypress, TX (US); Jhon Henry Trujillo Montenegro, Cali (CO); Jennifer Melissa Russi Castillo, Manizales (CO); Nestor Quevedo Cubillos, Bogota (CO)

(73) Assignee: AXURE TECHNOLOGIES S.A., Bogota (CO)

(21) Appl. No.: 14/359,138

(22) PCT Filed: Nov. 15, 2012
(86) PCT No.: PCT/US12/65237

§ 371 Date: May 19, 2014

Related U.S. Application Data
(60) Provisional application No. 61/561,857, filed on Nov. 19, 2011.

(51) Int. Cl. G01N 21/64; G01N 33/28

(52) U.S. Cl. CPC G01N 21/64; G01N 33/28
USPC 250/459.1; 250/458.1; 250/227.11

(57) ABSTRACT

Described herein are systems and methods for detecting and determining the presence and grade of petroleum oil in a sample. The methods and systems use the fluorescence produced by one or more transition metals present in the oil to detect and determine the presence and grade of the oil in the sample. The fluorescence produced by the metals is also useful as marker for tracking presence oil in the soil. The methods and offset the problems of the existing exploration technologies described above mentioned. Additionally, the methods and systems can be useful in identifying other compounds related to petroleum oil, such as carbon, hydrogen, and other types of aromatic compounds.

### CROSS REFERENCE TO RELATED APPLICATION

This application claims priority upon U.S. Provisional Application Ser. No. 61/561,857, filed Nov. 19, 2011. The application is hereby incorporated by reference in its entirety for all of its teachings.

### BACKGROUND

Exploration of petroleum oil is a very costly and uncertain process that is time-consuming and technology-labor intensive, which typically requires a large capital investment. This complex process requires technologies that target trace petroleum oil in order to find the real source of petroleum oil. The existing or conventional methods are based on the comparison of existing images or indirect technologies. There are other methods for identification of petroleum oil, using a fiber. Moreover, existing methods do not establish detection of petroleum based on the types of petroleum oil, which can introduce wide range of errors due to cross-reaction in the fluorescence. The existing rapid optical Screening tool (ROST)” is based on the use of a laser beam and monochromatic light. Although it is able to detect some chemical characteristics of the hydrocarbon and/or physical properties of the petroleum oil, it does not identify or detect quantitatively presence of petroleum oil based on metal fluorescence.

### SUMMARY

Described herein are systems and methods for detecting and determining the presence and grade of petroleum oil in a sample. The methods and systems use the fluorescence produced by one or more transition metals present in the oil to detect and determine the presence and grade of the oil in the sample. The fluorescence produced by the metals is also useful as marker for tracking presence oil in the soil. The methods and offset the problems of the existing exploration technologies described above mentioned. Additionally, the methods and systems can be useful in identifying other compounds related to petroleum oil, such as carbon, hydrogen, and other types of aromatic compounds.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a block diagram illustrating an example of the network environment for the petroleum detection system described herein.

FIG. 2 is a block diagram illustrating an example of a server utilizing the petroleum detection system described herein, as shown in FIG. 1.

FIG. 3 is a flow chart illustrating an example of the operation of the petroleum detection system described herein utilized by the server, as shown in FIG. 2.

FIG. 4 is a flow chart illustrating an example of the operation of the library construction process on the server that is utilized in the petroleum detection system described herein, as shown in FIGS. 2A-3.

FIG. 5 is a flow chart illustrating an example of the operation of the petroleum analysis process on the server that is utilized in the petroleum detection system described herein, as shown in FIGS. 2A-3.

FIG. 6 is a screen shot illustrating an example of the results of the operation of the petroleum analysis process on the server that is utilized in the petroleum detection system described herein, as shown in FIGS. 2, 3 and 5.

FIG. 7 shows samples of two types of soil used for in the experiments in the Examples.

FIG. 8 shows sandy soil sample partially impregnated with oil. Left-UV Radiation. Right- Halogen lighting. Sandy soil sample mixed with oil (Castilla crude) of 15 API. On the left, the same treatment under UV light at 366 nm. The soil that has been impregnated with oil shows intense fluorescence (top of the picture). In contrast, in the bottom of the picture, the section of land that has not been impregnated with oil shows no fluorescence.

FIG. 9 shows sandy soil samples impregnated with oil in the right section of the petri dish and soaked with oil and sludge from the oil activity in the left side of the box. Left—UV Radiation. Right—Halogen lighting. The photo on the right has in the right section of the petri dish a type of crude oil impregnated sediment, and the left is the same type of soil with crude oil mixed with a type of mud from the oil exploration. This sample was made to determine the incidence of mud (pollutant) in soil samples in order to have a control sample as well as to understand fluorescence disappearance or increase, which generates a false positive. The left side shows the same plate under UV light. The right section of this image shows a high fluorescence, whereas in the left section heterogeneous fluorescence is seen.

FIG. 10 shows three different soil samples taken from the well between 5,000 and 7,400 feet deep. To the right, well soil from 7,400 feet deep showed some oil traces. In the middle picture, well soil from 6,400 feet deep showed loss of fluorescence. Lastly, the sample on the left, soil from the same well and which was extracted at 5,000 feet deep showed no fluorescence.

FIG. 11 shows soil samples with Fe in various concentrations. Left—0% metal. Center—10 ppm Fe. Right—Fe of 100 ppm. Concentration with higher fluorescence was observed at 100 ppm. The sample of 0 ppm was used as negative control when no fluorescence.

FIG. 12 shows soil samples with a mixture of nickel, iron, and vanadium in various concentrations. The left control sample without metal; the right soil mixture with 100 ppm of Fe, Ni and V; and the center is a plate with 10 ppm of metals tested. At 100 ppm the metal distribution is less homogeneous but the points of fluorescence are in greater proportion or better defined, while the center plate fluorescence was observed with better distribution of the fluorescence but with less proportion. The plaque on the left with 0% metals has fluorescence different than the expressed in the other two plates, which is more similar to contamination fluorescence.

FIG. 13 shows an exemplary reactor for sediment conditions simulation for controlled environments.

FIG. 14 shows sample organization in the reactors using three different types of soil and combinations to simulate soil composition based on analysis of soils during excavation in Ecopetrol facilities in Barranca Bermeja Santander.

FIG. 15 shows samples taken from the reactor obtained by small windows in each of the reactors. After sample extraction, these were immediately washed with dilute nitric acid and then exposed for UV-Vis analysis.

FIG. 16 shows samples at 10 ppm of Fe with and without oil extracted from the reactors. Known soil mix at 10 ppm of iron. Crude oil impregnated sample and iron without crude oil sample were analyzed. In both plates fluorescence was observed although in the sample containing oil the fluorescence was better defined and more abundant.

FIG. 17 shows samples at 50 ppm of Fe with and without oil extracted from the reactors.

FIG. 18 shows samples at 10 ppm Ni with and without oil extracted from the reactors.

FIG. 19 shows samples at 50 ppm Ni with and without oil extracted from the reactors.

FIG. 20 shows samples taken in the field exposed to UV light. Ground samples were illuminated with UV light. In the center image, some fluorescence did not belong to the one expressed by the metal, as it was a reddish fluorescence, while the metal is between green and blue (expressed between 400 nm and 515 nm). In the right image fluorescence was not observed.

FIG. 21 shows soil samples impregnated with different API oil exposed to UV light. The left image shows a mixture of soil with crude oil, which contained only crude oil traces and a generous amount of soil. In the middle image there are three types of soil, with 0.5 ml of 15 API crude oil added per 5 g of soil after 24 hours. The right image sample was treated with 25 API crude oil. Fluorescence increased as the API degree increased. The lighter the API, the greater the oil is distributed is in the soil, compared with the sample analyzed in FIG. 20.

FIG. 22 shows the UV spectrum obtained from the analysis of soil without metal obtained with a spectrometry device equipped with optic fiber sensor. The purple line between 380 and 405 nm corresponds to radiation from the light source that emits light at 366 nm. Here, there is no fluorescence expressed by the sample.

FIG. 23 shows soil with 10 ppm vanadium and 15 API crude oil analysis, with blue-violet fluorescence expressed by vanadium between 405 nm and 430 nm at high intensity.

FIG. 24 shows soil with 50 ppm vanadium and 41 API crude oil analysis, with high expression of fluorescence intensity between 400 nm and 410 nm.

FIG. 25 shows soil with 50 ppm nickel and 15 API crude analysis, with blue and green fluorescence between 411 nm and 450 nm in a broad spectrum and high intensity indicating that it can identify lower metal concentrations.

FIG. 26 shows soil analysis with 10 ppm nickel and 41 API crude, where high fluorescence was observed between 420 nm and 450 nm.

FIG. 27 shows the correlation between the metal concentration and fluorescence. The following equation was used in the correlation with R²=0.92. Results are shown in Table 3.

$$\text{?} ( x ) \text{?} [ \text{?} ] \text{?} ∀ ( x , y \geq \text{?} \text{?} \leq \text{?} ) \text{?} \text{?} \text{?} ( x ) \text{?} . \\ \text{?} \text{indicates text missing or illegible when filed}$$

Because of this low correlation, a relationship was found using the neural system that would allow greatest degree of correlation between metal concentrations and fluorescence. The values are normalized in equation 1. (Normalization values Max=600, Min=0).

FIG. 28 shows the identification of metals. Metal Type Vs Sample No. (Entry pattern). Vanadium [50, 150], Copper (150.250], nickel [250, 350], Fe [350.450). JpredictionPetrolium general picture. Training and testing results in the computational model. Approx Number of patterns=500 data presented in training (see Table 5).

FIG. 29 shows the metal abundance levels prediction and identification. Normalized values with the equation No. 1. Ranks for Standardization [max=0, min=700]. [0, 0.0001]=Level 1, (0.0001, 0.0002]=Level 2, (0.0002, 0.0003)=Level 3, (0. 0003, 0.0004]=Level 4, (0.0004, 0.0005]=Level 2, (0.0005, 0.0006]=Level 6 (0. 0006, 0.0007]=Level 7. jpredictionPetrolium generated image. Training and testing results shown by computational model. Approx Number of patterns=500 data presented in training. The graphic has been amplified in thousands for better viewing (see Table 4).

FIG. 30 shows the computational model representation with fluorescent imaging. Inputs=Fluorescence Data, Outputs=fluorescence level. Neuronal Model Representation=internal neural networks system which allow training and evaluation of new results to determine the output

FIG. 31 shows the soil sample collected in the field soaked with oil of 15 API prepared in the laboratory for comparison to samples without rude.

FIG. 32 shows the soil sample collected in the field soaked with oil of 21 API prepared in the laboratory with oil traces. Different types of soil generate different fluorescence with more or less intensity, containing the same amount of oil.

FIG. 33 shows the soil sample collected from the field and soaked with oil of 30 API petroleum oil. The sample emitted fluorescence with some red-brownish specks due to higher iron concentration with less nickel and vanadium.

FIG. 34 shows soil samples collected in the field soaked with oil of 41 API fluoresce very intensely, although the amount of oil impregnated is very low and the soaking time was short. This indicates that if oil goes through soil and leaves its mark can be easily determined with this system.

FIG. 35 shows soil samples collected in the field soaked with oil of 47 API. This is one of the lighter oils, which could permeate the soil more easily. This allowed it to come in contact with soil metals increasing fluorescence.

FIG. 36 shows crude samples exposed to UV radiation at 366 nm emitting fluorescence. The crude samples of 15 and 21 API emitted no fluorescence; however, fluorescence was observed when they came in contact with soil and when the reaction time allowed metals to react with hydrocarbons (FIGS. 31 and 32). 30 API crude fluorescence shows low intensity even when it came in contact with soil remained low (FIG. 33).

FIG. 37 shows soil samples without metal presence and does not emit any fluorescence. The soil sample was washed with HNO₃ to prevent the presence of hydrocarbon fluorescence and if there is a considerable concentration of metal in it to fluoresce and be able to quantify it and enter it to the model to improve behavior of control sample.

FIG. 38 shows soil samples treated with a Ni solution exposed to UV radiation of 366 nm expressing fluorescence. The soil sample was collected and washed with HNO₃ to prevent hydrocarbon fluorescence and to help better metals fluorescence response.

FIG. 39 shows soil samples with Ni at 50 ppm impregnated with oil of 15 API exposed to UV radiation at 366 nm, expressing fluorescence obtained from the reactor prepared in the laboratory, and washed with dilute nitric acid.

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, compounds, compositions, methods, conditions, or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a transition metal” includes two or more metals, and the like.

“Optional” or “optionally” means that the subsequently described event or circumstance can or cannot occur, and that the description includes instances where the event or circumstance occurs and instances where it does not.

Described herein are systems and methods for determining the presence and grade of petroleum in a sample. In one aspect, the method is embodied in a computer program product for execution on an instruction processing system, comprising a tangible storage medium readable by the instruction processing system and storing instructions for execution by the instruction processing system for performing the method comprising:

a) obtaining a sample that may or may not contain petroleum; and
b) detecting the presence of fluorescence produced by the sample, wherein the presence of fluorescence indicates the presence of petroleum in the sample.

In one aspect, the method is embodied in a computer program product for execution on an instruction processing system, comprising a tangible storage medium readable by the instruction processing system and storing instructions for execution by the instruction processing system for performing the method comprising:

a) obtaining a sample comprising petroleum;
b) quantifying the amount of fluorescence produced by the petroleum in the sample;
c) comparing the amount of fluorescence in the sample to a standard curve, where the curve correlates the amount of fluorescence to the grade of petroleum; and
d) identifying the grade of petroleum in the sample.

In general, a petroleum detection system is described herein that can process fluorescence data produced by a sample of oil when the sample is exposed to UV light and correlate this data to the type and grade of oil present in the sample. The computer program is applicable on all remote devices connected to a server hosting the teledermatology systems and methods described herein. While described below with respect to a single computer, the system and method for petroleum detection system is typically implemented in a networked computing environment in which a number of computing devices communicate over a local area network (LAN), over a wide area network (WAN), or over a combination of both LAN and WAN.

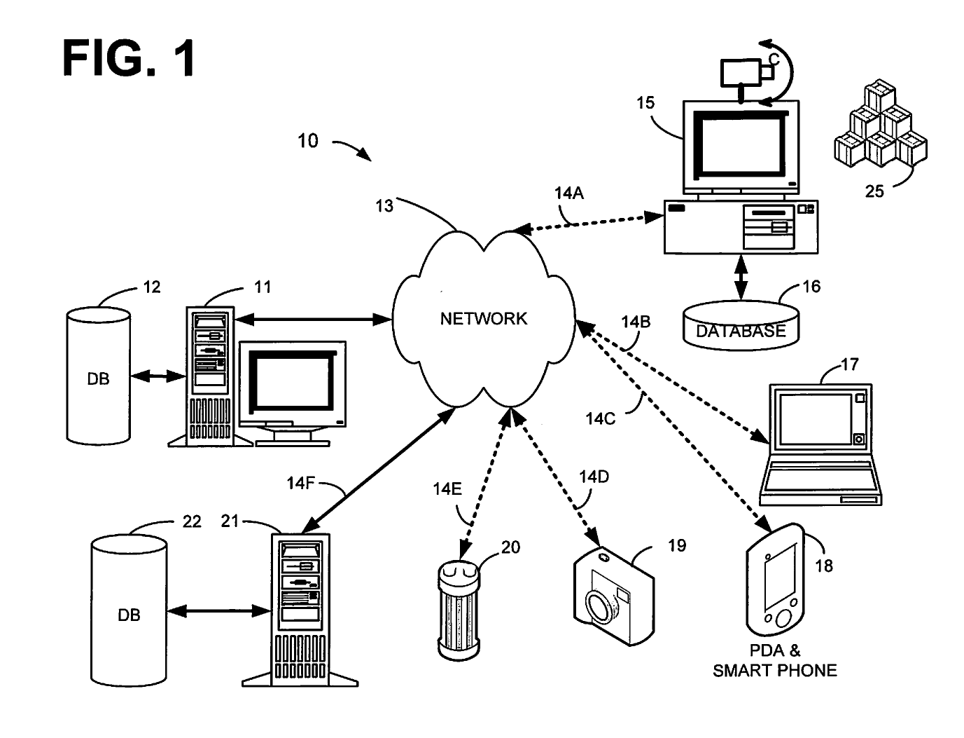

Referring now FIGS. 1-6, in which like numerals illustrate like elements throughout the several views. FIG. 1 illustrates an example of the basic components of a system 10 using the petroleum detection system. The system 10 includes a server 11 and the remote devices 15, 17 and 18 that utilize the petroleum detection system.

Each remote device 15, 17 and 18 has applications and can have a local database 16. Server 11 contains applications, and a database 12 that can be accessed by remote device 15, 17 and 18 via connections 14(A-C), respectively, over network 13. The server 11 runs administrative software for a computer network and controls access to itself and database 12. The remote device 15, 17 and 18 may access the database 12 over a network 13, such as but not limited to: the Internet, a local area network (LAN), a wide area network (WAN), via a telephone line using a modem (POTS), Bluetooth, WiFi, cellular, optical, satellite, RF, Ethernet, magnetic induction, coax, RS-485, the like or other like networks. The server 11 may also be connected to the local area network (LAN) within an organization (i.e. a hospital or medical complex).

The remote device 15, 17 and 18 may each be located at remote sites. Remote device 15, 17 and 18 include but are not limited to, PCs, workstations, laptops, handheld computer, pocket PCs, PDAs, pagers, WAP devices, non-WAP devices, cell phones, palm devices, printing devices and the like. Included with each remote device 15, 17 and 18 is an ability to obtain images of the material being analyzed. In the remote device 15, there is a special camera 24 for capturing images of material being analyzed 25. In remote devices 17 and 18, they are maybe integrated cameras for acquiring images of the material being analyzed or the ability to download photographs of material being analyzed 25 in a digital form. Digital camera 19 captures digital photographs of the samples, which enables the digitization of images for building a baseline library and the further analysis of samples.

Thus, when a user at one of the remote devices 15, 17 and 18 desires to access petroleum detection status from the database 12 at the server 11, the remote device 15, 17 and 18 communicates over the network 13, to access the server 11 and database 12.

Third party vendors computer systems 21 and databases 22 can be accessed by the petroleum detection system 100 on server 11 in order to access other analyzed materials and provide analytics. Data that is obtained from third party vendors computer system 21 and database 22 can be stored on server 11 and database 12 in order to provide later access to the user on remote devices 15, 17 and 18. It is also contemplated that for certain types of data that the remote devices 15, 17 and 18 can access the third party vendors computer systems 21 and database 22 directly using the network 13.

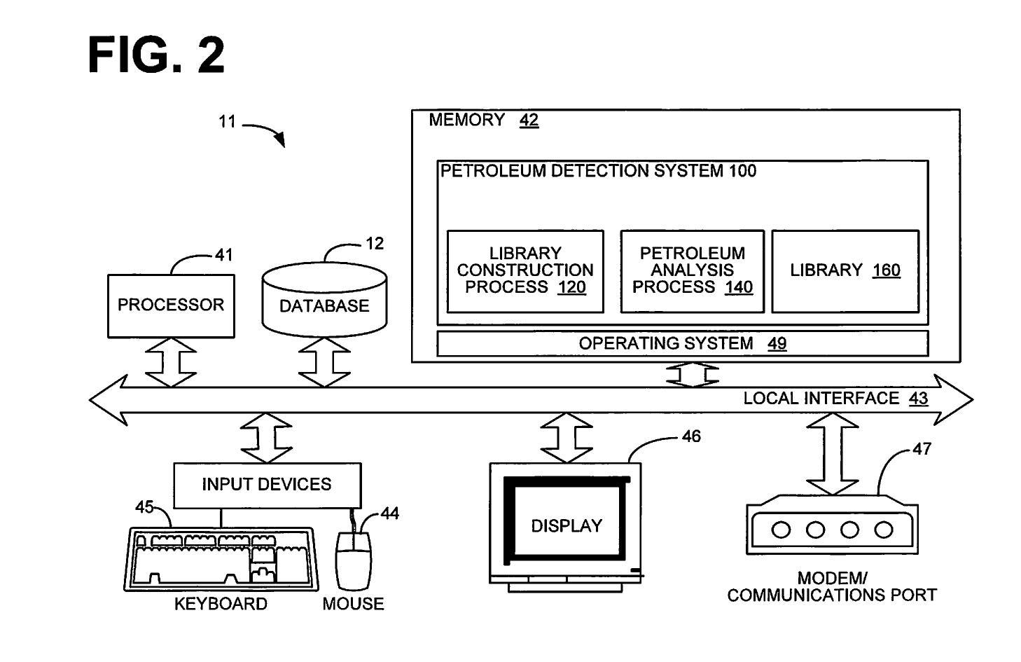

Illustrated in FIG. 2 is a block diagram demonstrating an example of server 11, as shown in FIG. 1, utilizing the petroleum detection system 100 described herein. Server 11 includes, but is not limited to, PCs, workstations, laptops, PDAs, palm devices and the like. The processing components of the third party vendors computer systems 21 and remote devices 15, 17 and 18 are similar to that of the description for the server 11 (FIG. 2).

Generally, in terms of hardware architecture, as shown in FIG. 2, the server 11 includes a processor 41, memory 42, and one or more input and/or output (I/O) devices (or peripherals) that are communicatively coupled via a local interface 43. The local interface 43 can be, for example but not limited to, one or more buses or other wired or wireless connections, as is known in the art. The local interface 43 may have additional elements, which are omitted for simplicity, such as controllers, buffers (caches), drivers, repeaters, and receivers, to enable communications. Further, the local interface 43 may include address, control, and/or data connections to enable appropriate communications among the aforementioned components.

The processor 41 is a hardware device for executing software that can be stored in memory 42. The processor 41 can be virtually any custom made or commercially available processor, a central processing unit (CPU), data signal processor (DSP) or an auxiliary processor among several processors associated with the server 11, and a semiconductor based microprocessor (in the form of a microchip) or a macroprocessor.

Examples of suitable commercially available microprocessors are as follows: an 80x86 or Pentium series microprocessor from Intel Corporation, U.S.A., a PowerPC microprocessor from IBM, U.S.A., a Sparc microprocessor from Sun Microsystems, Inc, a PA-RISC series microprocessor from Hewlett-Packard Company, U.S.A., or a 68xxx series microprocessor from Motorola Corporation, U.S.A.

The memory 42 can include any one or combination of volatile memory elements (e.g., random access memory (RAM, such as dynamic random access memory (DRAM), static random access memory (SRAM), etc.)) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 42 may incorporate electronic, magnetic, optical, and/or other types of storage media. Note that the memory 42 can have a distributed architecture, where various components are situated remote from one another, but can be accessed by the processor 41.

The software in memory 42 may include one or more separate programs, each of which comprises an ordered listing of executable instructions for implementing logical functions. In the example illustrated in FIG. 2A, the software in the memory 42 includes a suitable operating system (O/S) 49 and the petroleum detection system 100 described. As illustrated, the petroleum detection system 100 of the present invention comprises numerous functional components including, but not limited to, the library construction process 120, petroleum analysis process 140 and library 160.

A non-exhaustive list of examples of suitable commercially available operating systems 49 is as follows (a) a Windows operating system available from Microsoft Corporation; (b) a Netware operating system available from Novell, Inc.; (c) a Macintosh operating system available from Apple Computer, Inc.; (e) a UNIX operating system, which is available for purchase from many vendors, such as the Hewlett-Packard Company, Sun Microsystems, Inc., and AT&T Corporation; (d) a LINUX operating system, which is freeware that is readily available on the Internet; (e) a run time Vxworks operating system from WindRiver Systems, Inc.; or (f) an appliance-based operating system, such as that implemented in handheld computers or personal data assistants (PDAs) (e.g., Symbian OS available from Symbian, Inc., PalmOS available from Palm Computing, Inc., and Windows CE available from Microsoft Corporation).

The operating system 49 essentially controls the execution of other computer programs, such as the petroleum detection system 100, and provides scheduling, input-output control, file and data management, memory management, and communication control and related services. However, it is contemplated by the inventors that the petroleum detection system 100 is applicable on all other commercially available operating systems.

The petroleum detection system 100 may be a source program, executable program (object code), script, or any other entity comprising a set of instructions to be performed. When a source program, then the program is usually translated via a compiler, assembler, interpreter, or the like, which may or may not be included within the memory 42, so as to operate properly in connection with the O/S 49. Furthermore, the petroleum detection system 100 can be written as (a) an object oriented programming language, which has classes of data and methods, or (b) a procedure programming language, which has routines, subroutines, and/or functions, for example but not limited to, C, C++, C#, Pascal, BASIC, API calls, HTML, XHTML, XML, ASP scripts, FORTRAN, COBOL, Perl, Java, ADA, .NET, and the like.

The I/O devices may include input devices, for example but not limited to, a mouse 44, keyboard 45, scanner (not shown), microphone (not shown), etc. Furthermore, the I/O devices may also include output devices, for example but not limited to, a printer (not shown), display 46, etc. Finally, the I/O devices may further include devices that communicate both inputs and outputs, for instance but not limited to, a NIC or modulator/demodulator 47 (for accessing remote devices, other files, devices, systems, or a network), a radio frequency (RF) or other transceiver (not shown), a telephonic interface (not shown), a bridge (not shown), a router (not shown), etc.

If the server 11 is a PC, workstation, intelligent device or the like, the software in the memory 42 may further include a basic input output system (BIOS) (omitted for simplicity). The BIOS is a set of essential software routines that initialize and test hardware at startup, start the O/S 49, and support the transfer of data among the hardware devices. The BIOS is stored in some type of read-only-memory, such as ROM, PROM, EPROM, EEPROM or the like, so that the BIOS can be executed when the server 11 is activated.

When the server 11 is in operation, the processor 41 is configured to execute software stored within the memory 42, to communicate data to and from the memory 42, and generally to control operations of the server 11 are pursuant to the software. The petroleum detection system 100 and the O/S 49 are read, in whole or in part, by the processor 41, perhaps buffered within the processor 41, and then executed.

When the petroleum detection system 100 is implemented in software, as is shown in FIG. 2, it should be noted that the petroleum detection system 100 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, propagation medium, or other physical device or means that can contain or store a computer program for use by or in connection with a computer related system or method.

More specific examples (a nonexhaustive list) of the computer-readable medium would include the following: an electrical connection (electronic) having one or more wires, a portable computer diskette (magnetic or optical), a random access memory (RAM) (electronic), a read-only memory (ROM) (electronic), an erasable programmable read-only memory (EPROM, EEPROM, or Flash memory) (electronic), an optical fiber (optical), and a portable compact disc memory (CDROM, CD R/W) (optical). Note that the computer-readable medium could even be paper or another suitable medium, upon which the program is printed or punched (as in paper tape, punched cards, etc.), as the program can be electronically captured, via for instance optical scanning of the paper or other medium, then compiled, interpreted or otherwise processed in a suitable manner if necessary, and then stored in a computer memory.

In an alternative embodiment, where the petroleum detection system 100 is implemented in hardware, the petroleum detection system 100 can be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The remote devices 15, 17 and 18 provides access to the petroleum detection system 100 on server 11 and database 12 using the remote device system, including for example, but not limited to an Internet browser. The information accessed in server 11 and database 12 can be provided in the number of different forms including but not limited to ASCII data, WEB page data (i.e. HTML), XML or other type of formatted data.

Included with each remote device 15, 17 and 18 is an ability to obtain images of the client. In the remote device 15, there is a camera 24 for capturing images of client 20. In remote devices 17 and 18, they are maybe integrated cameras for acquiring images of the client or the ability to download photographs of client 20 in a digital form.

As illustrated, the remote device 15, 17 and 18 and 21 are similar to the description of the components for server 11 described with regard to FIG. 2. Hereinafter, the remote devices 15, 17 and 18 that will be referred to as remote devices 15 for the sake of brevity.

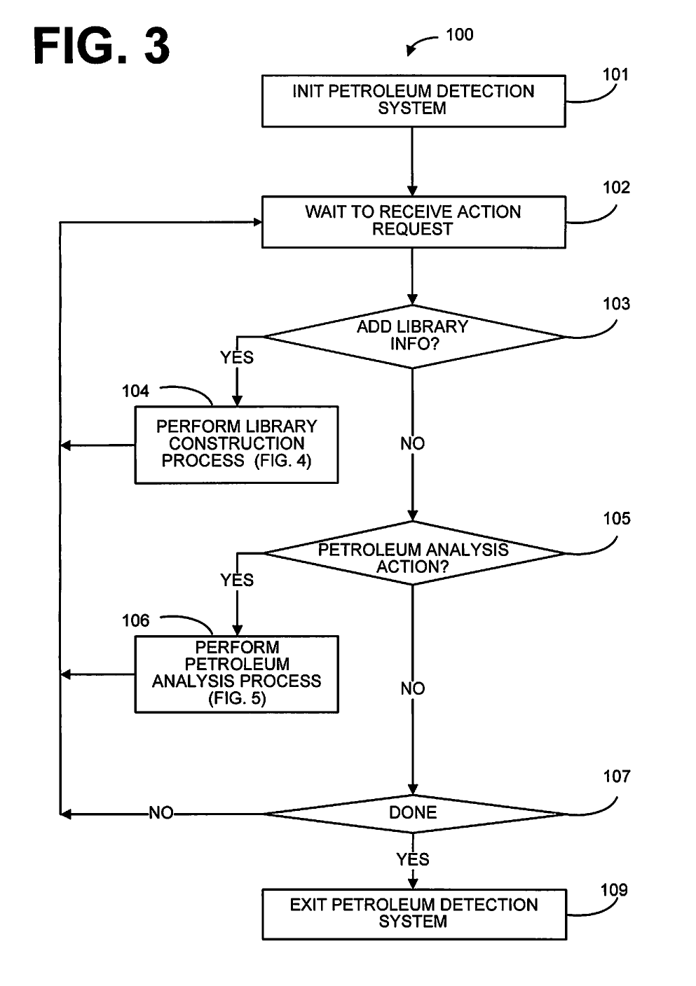

FIG. 3 is a flow chart illustrating an example of the operation of the petroleum detection system 100 described herein utilized by the server 11, as shown in FIG. 2. The petroleum detection system 100 detects the presence of petroleum present in different medium.

First at step 101, the petroleum detection system 100 is initialized. This initialization includes the startup routines and processes embedded in the BIOS of the server 11. The initialization also includes the establishment of data values for particular data structures utilized in the petroleum detection system 100.

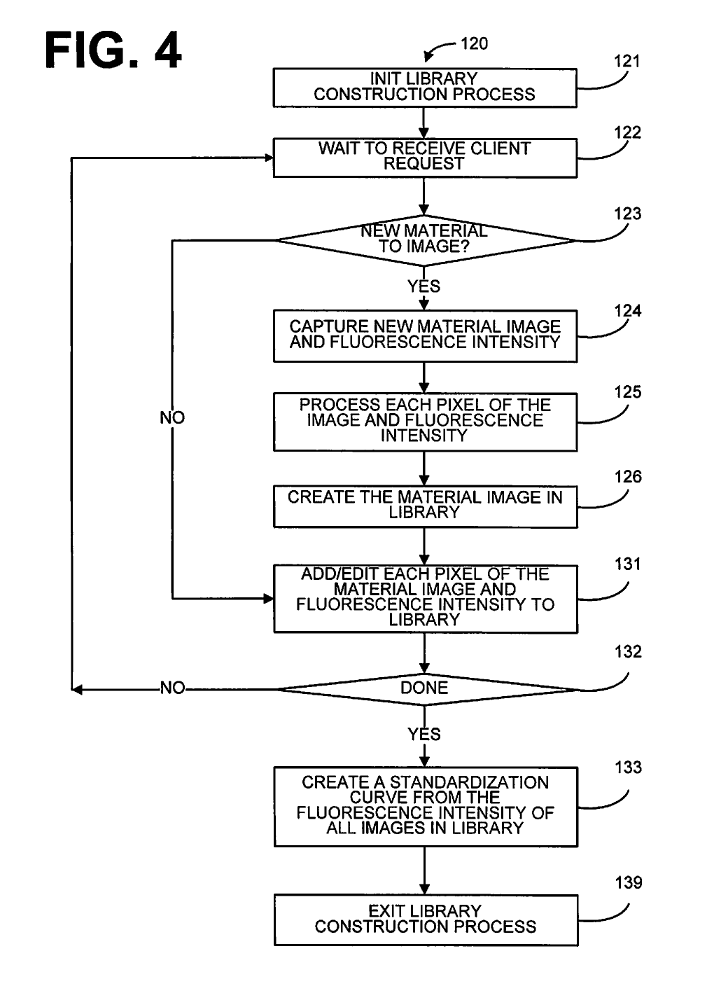

At step 102, the petroleum detection system 100 waits to receive an action request. Once an action is received at step 102, it is determined if the action is to add a material sample to the library 160 at step 103. If it is determined that the action is not to add a new material sample to the library 160, then the petroleum detection system 100 skips step 105. However, if it is determined in step 103 that a new material sample is to be added to the library 160, then the petroleum detection system 100 performs the library construction process at step 104. The library construction process is herein defined in further detail with regard to FIG. 4. After performing the library construction process, the petroleum detection system 100 returns to step 102.

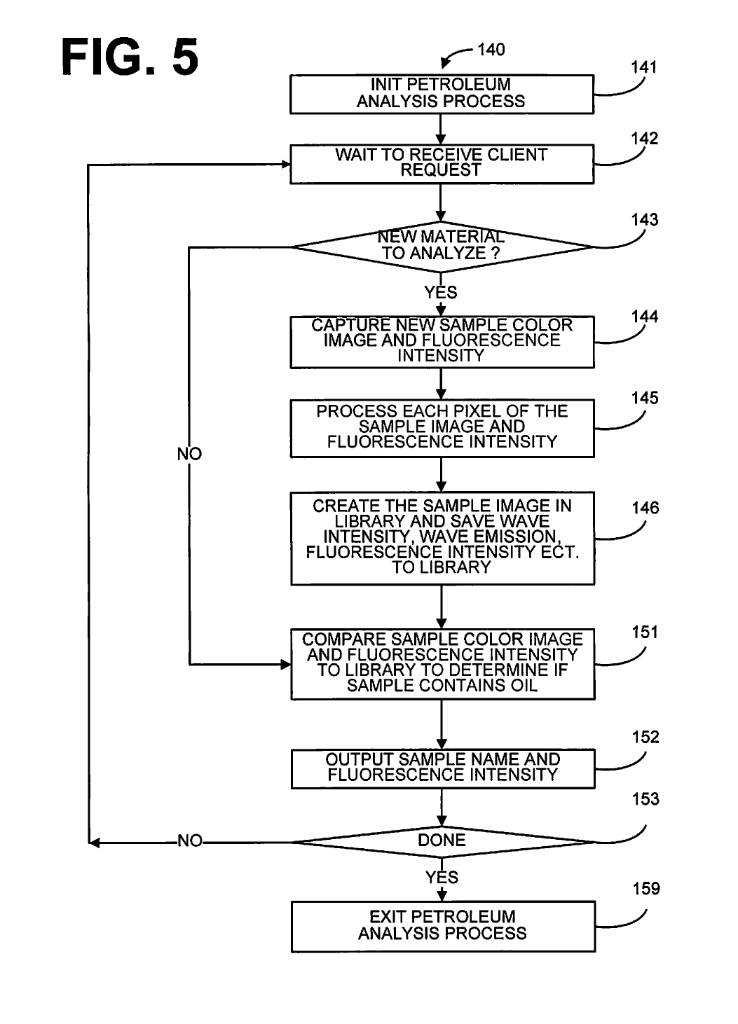

At step 105, it is determined if the action is a petroleum analysis action. If it is determined that the action is not a petroleum analysis action, then the petroleum detection system 100 skips step 107. However, if it is determined in step 105 that it is a petroleum analysis action, then the petroleum detection system 100 performs the petroleum analysis process at step 106. The petroleum analysis process is herein defined in further detail with regard to FIG. 5. After performing the petroleum analysis process, the petroleum detection system 100 returns to step 102.

At step 107, it is determined if the petroleum detection system 100 is to wait for an additional action request. If it is determined at step 107 that the petroleum detection system is to wait to receive additional actions, then the petroleum detection system 100 returns to repeat steps 102 through 107. However, if it is determined at step 107 that there are no more actions to be received, then the petroleum detection system 100 then exits at step 109.

FIG. 4 is a flow chart illustrating an example of the operation of the library construction process 120 on the server that is utilized in the petroleum detection system 100, as shown in FIGS. 2A-3. The library construction process 120 establishes or modifies specific information residing in library 160 (FIG. 2). Once the new material information is placed in server 11, it is available for creating the standardization curve and petroleum analysis. A brief overview of one exemplary process is as follows: 1) waits to receive a client configure request; 2) determine if the material is a new material; 3) process each pixel in the materials image; 4) upload new/modify existing material information from local machine; and 5) done.

First at step 121, the library construction process 120 is initialized. This initialization includes the startup routines and processes embedded in the BIOS of the server 11. The initialization also includes the establishment of data values for particular data structures utilized in the library construction process 120.

At step 122, the library construction process 120 waits to receive a new client request. Once a new client request has been received, the library construction process 120 determines if the material is a new material to the petroleum detection system 100. If it is determined at step 123 that the material is not a new material, then the library construction process 120 skips step 131 to enable the material to enter new or edit existing material data. However, if it is determined at step 123 that the material is a new material, then the library construction process 120 captured the new materials image and fluorescence intensity at step 124. At step 125, each pixel in the image of the new material is processed along with its fluorescence intensity. In this aspect, the fluorescence emitted by the sample can be photographed with a digital camera.

In one embodiment, step 125 in FIG. 4, a series of samples containing petroleum oil with known grades and amounts of transition metals that fluoresce when exposed to UV light can photographed with a digital camera. Each photograph produces a library that permits the processing of a set of images that can be digitized. In one aspect, the library was developed using Anci C programming language (library “OpenGL”) and the programming Python language (library “Image”). The library “OpenGL” and “Image” permit the processing of the pixels for each image for each sample. The library “OpenGL” is a library designed in C language, which connects the library with the computational model. The data is each pixel of the processed image. The library “Image” permits the reading of the pixels of the image in order to create a plain file with information from each of images. Exemplary methods for generating libraries of pixel data for a plurality of samples is provided in the Examples.

At step 131, the library construction process 120 enables the addition of new image information or editing existing material information in the new material record. For example, in order to enhance the ability of the petroleum detection system 100 system to detect petroleum oil in a sample, an optical fiber optical system can be used to optimize the standardization curve 133. Here, the fiber optic system is more accurate than using a camera and pictures with respect to detecting and quantifying fluorescence produced by the metals present in the oil. Referring to FIG. 1, the optical fiber sensor 20 detects fluorescence emitted by the sample having a known metal concentration and grade of oil. In case, of false positive (i.e., soil samples with fluorescence but containing petroleum), the difference in fluorescence between the real positive sample and the false positive sample determines the real value of the fluorescence to be include in the standardization curve. This will eliminate the noise (interference) between real value and the false value. The fluorescence data is fed into the petroleum detection system in order to optimize the standardization curve 133. The Examples provide procedures for using an optical fiber sensor to collect fluorescence data that is used in the petroleum detection systems described herein.

At step 132, it is determined if the library construction process 120 is to wait for additional client requests. If it is determined at step 132 that the library construction process 120 is to wait for additional client requests, then the library construction process 120 returns to repeat steps 122 through 132. However, if it is determined at step 132 that there are no more client actions to be received, then the library construction process 120 create a current standardization curve from the fluorescence intensity of all analyze material images in the library 160. After creating the new standardization curve, the library construction process 120 then exits at step 139.

FIG. 5 is a flow chart illustrating an example of the operation of the petroleum analysis process 140 on the server that is utilized in the petroleum detection system 100, as shown in FIGS. 2 and 3. Once the new fluorescence data from the sample is introduced to server 11, it is available for petroleum analysis. A brief overview of one exemplary process is as follows: 1) wait to receive a quiet request for petroleum analysis; 2) determine if the material (i.e., fluorescence) to be analyzed is new; 3) acquire new sample image and data regarding the new sample; 4) process each pixel in the new sample image; 5) create a new sample image in library 160; 6) compared the new sample color image and fluorescence intensity to materials in library 162 determine if the sample contains oil; and 7) output the sample name, fluorescence intensity, and, where applicable, the grade of the petroleum oil.

First at step 141, the petroleum analysis process 140 is initialized. This initialization includes the startup routines and processes embedded in the BIOS of the server 11. The initialization also includes the establishment of data values for particular data structures utilized in the petroleum analysis process 140.

At step 142, the petroleum analysis process 140 waits to receive a client transaction requesting sample analysis. Once a client transaction requesting sample analysis has been received, the petroleum analysis process 140 then determines if the material to be analyzed is a new sample at step 143. If the material to be analyzed is not a new sample, then the petroleum analysis process 140 skips step 151. However, if the material to be analyzed is a new sample, then the new samples color image and fluorescence intensity is captured at step 144.

At step 145, the fluorescence intensity produced by the sample is processed. The digital camera 19 or optical fiber sensor 20 can be used to measure the fluorescence intensity, where the optical fiber sensor 20 is preferred due to its greater sensitivity. At step 146, a new record is created for the new sample in library 160 and information for the new sample is saved. This information saved includes but is not limited to the way the intensity, wave the missions, florescent intensity and the like.

At step 151, the new sample color image and fluorescence intensity is compared to data in library 160 in order to determine if the new sample contains oil. This computer analysis would be much like the computerized analysis of Pap smears and other tissue cultures.

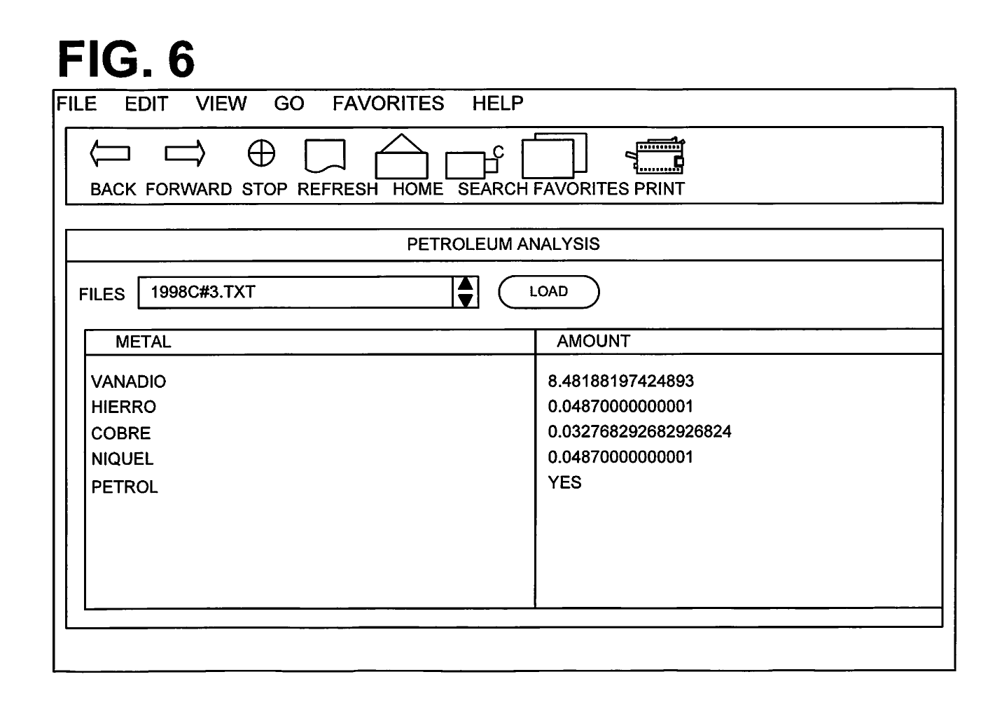

At step 152, the petroleum analysis process 140 outputs the sample name and fluorescence intensity of each material in the sample. An example of the information output is illustrated in FIG. 6.

At step 153, it is determined if the petroleum analysis process 140 is to wait for additional samples to be analyzed. If it is determined at step 153 that the petroleum analysis process 140 is to wait for additional client transactions, then the petroleum analysis process 140 returns to repeat steps 142 through 153. However, if it is determined at step 154 that there are no more samples to be analyzed, then the petroleum analysis process 140 then exits at step 159.

FIG. 6 is a screen shot illustrating an example of the results of the operation of the petroleum analysis process on the server that is utilized in the petroleum detection system 100 described herein, as shown in FIGS. 2, 3 and 5.

The petroleum detection system described herein is capable of detecting the presence of petroleum oil in a sample as well as the grade of the oil. Using the system discussed above and the techniques in the Examples, a test sample suspected of containing petroleum oil can be exposed to UV light, and the fluorescence produced by the sample can be detected and fed into the petroleum detection system. For example, the petroleum detection system can be “trained” to correlate the amount of fluorescence to the density of the petroleum oil per the American Petroleum Institute (API). The Examples provide procedures for training the petroleum detection system to correlate fluorescence values to API values in order to asses the type of oil present in the sample.

The petroleum detection system described herein is versatile in detecting oil in a number of different types of samples. If the oil sample contains at least one metal that fluoresces upon exposure to UV light and detectable by the optical sensor, then the computer program is effective in quantifying the amount of the metal that is present in the sample and identifying the type of oil. For example, the oil sample can contain vanadium, nickel, iron, copper, or any combination thereof. Each metal emits a different intensity or wavelength of fluorescence. Therefore, brighter fluorescence does not necessarily correspond to right values of petroleum. The petroleum detection system described herein takes this into account. The Examples provide numerous results where samples containing different types and amounts of metals were evaluated.

The sample tested using the petroleum detection system and methods described herein can be in any medium that may or may not contain petroleum oil. In one aspect, the sample comprises a soil sample, including but not limited to, sandy soil, sludge, clay sediment, sandy sediment, or any combination thereof. The Examples provide results from the testing of several different types of soil sample with respect to the detection of oil.

Any process descriptions or blocks in flow charts should be understood as representing modules, segments, or portions of code which include one or more executable instructions for implementing specific logical functions or steps in the process, and alternate implementations are included within the scope of the preferred embodiment of the present invention in which functions may be executed out of order from that shown or discussed, including substantially concurrently or in reverse order, depending on the functionality involved, as would be understood by those reasonably skilled in the art of the present invention.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

1. Sampling of Soil and/or Liquid Petroleum Oil

Sediment samples with oil associated metals (oxidized metals Ni, Fe, V) were prepared to confirm that the presence of these metals in the crude generated fluorescence. 37 samples with different metals concentrations were created in ranges from 0 to 100 ppm, with each metal separately and with a combination of them, taking into account the percentage of humidity. Soil samples containing the above metals were exposed to UV radiation at several wavelengths with a lamp of 250 nm to 366 nm. However, the wavelength of 366 nm was preferable. Under this irradiation, the increased expression of fluorescence was at 100 or over 100 ppm, with a relative humidity of 30% in sandy sediments.

A photographic record was generated, which enabled quantification of fluorescence to determine the relationship between intensity and concentration of the metal. Data were entered into the computational model (i.e., petroleum detection system) in order to train the model on the identification and differentiation of metals as well as to make predictions about the soil and subsoil from which samples are extracted.

The fluorescence emitted by the metal samples was generated by metals when exposed to UV light. The intensity generated was captured by a camera system (Nikon D80 features a 10.2-megapixel CCD sensor, Nikon DX format (23.6×15.8 mm with a 1.5× conversion factor)), with UV filter recording the fluorescence. Using photoluminescence software, fluorescence intensity was recorded by an automatic mathematical analysis that gave ranges of fluorescence intensity associated with the metal concentration.

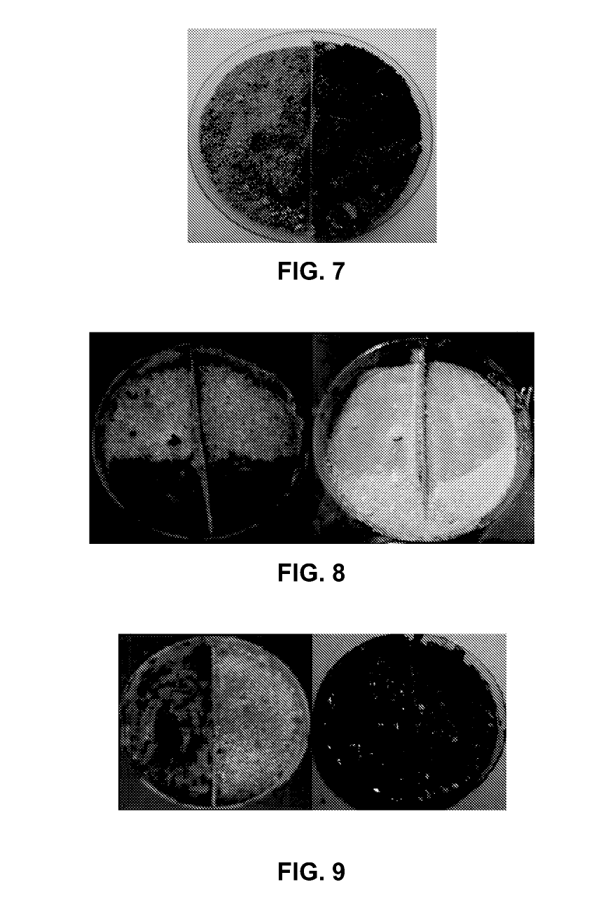

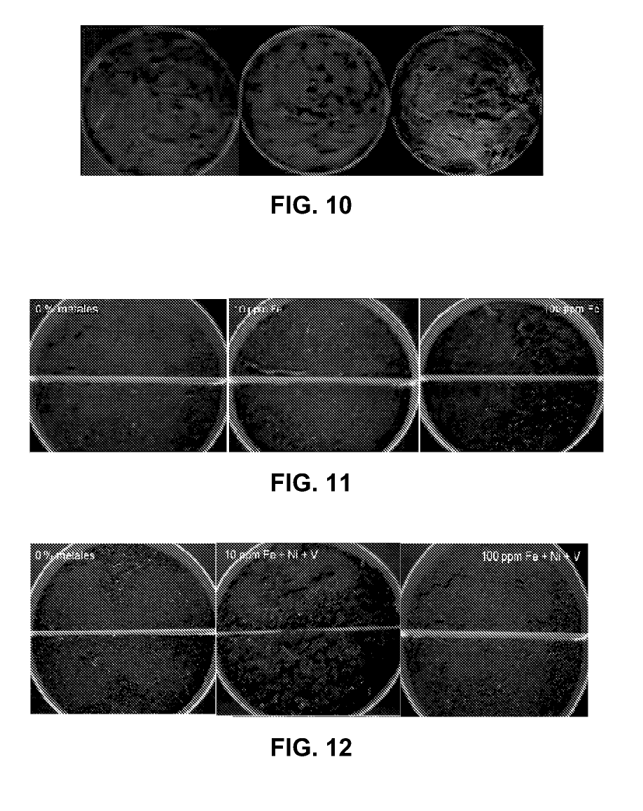

Furthermore, different sediment samples of different types (sandy, clay, conglomerates, and mixed) were used (FIG. 7). Although all emitted fluorescence, sandy soil emitted the most fluorescence. Samples impregnated with oil of 15 API and with different metals (Ni, Fe, V and Cu) associated with oil presence were prepared to demonstrate the relationship of fluorescence and oil traces, using as a form of excitation a UV radiation (λ 366 nm) and infrared.

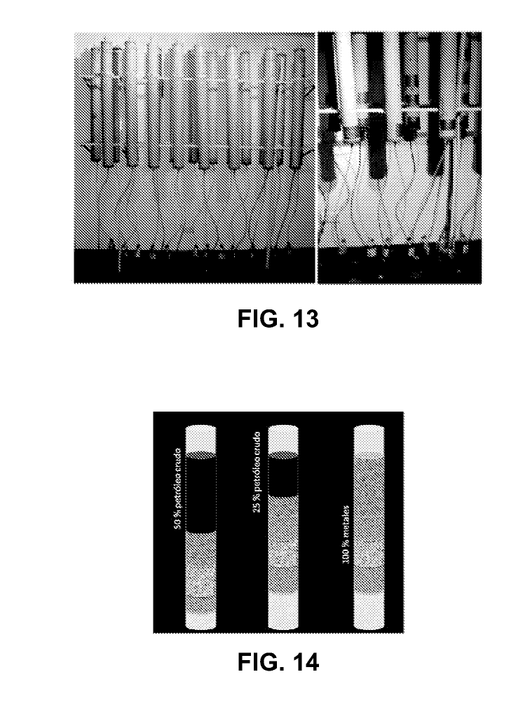

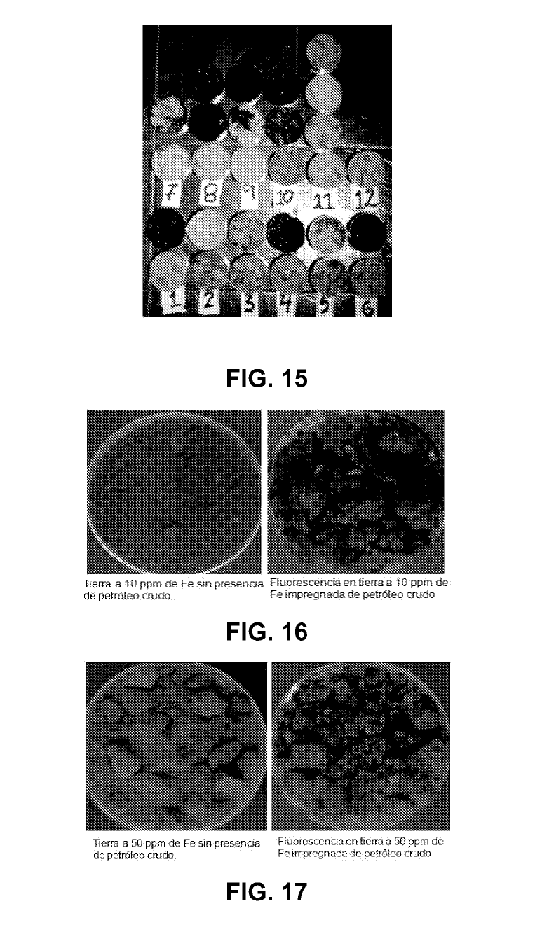

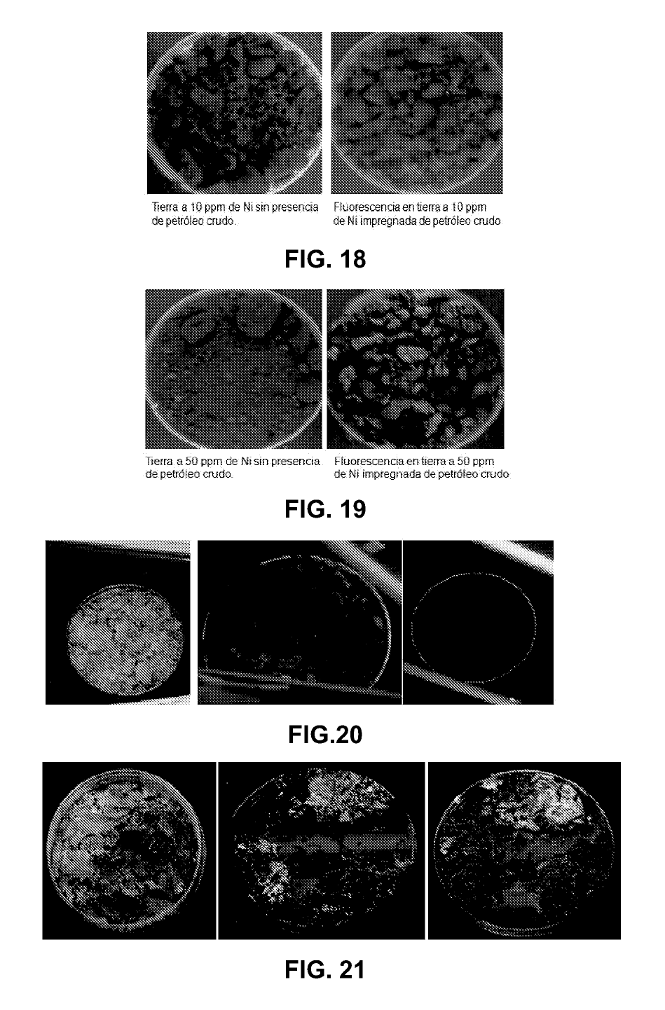

The reactor in FIG. 13 was used to generate samples simulating controlled conditions from deep underground. PVC pipes of 1 mt length of 10 cm in diameter were placed. Each tube was filled once sealing one of the ends, while the other served as input of the sediments. For each reactor, compositions of soil+metal at 0 ppm, 10 ppm, 50 ppm and 100 ppm of metal ratios 1:1, 1:2, 1:3 ratio were prepared.

The homogeneous mixture of sediment and metals solution mentioned above were introduced into the bottom of the reactor, distributing different types of sediment in a way that the distribution of soil layers encountered in well were simulated in proportion of: clay sediments, sandy sediments, washed sandy sediments, and conglomeratic sediments, 0:1:1:1, 1:2:1:1, 1:1:2:1, 0:1:1:2, 2:1:1:1, 1:3:2:1, 1:3:1:1, 1:2:3:0, 0:3:1:2, 2:0:3:1. Later 15 API crude was poured over the layers of sediment with an oil mixing ratio of: 1:1, 0.75:0.25, 1:0, respectively (FIG. 14), generating a pressurized flow of oil through sediments. In these experiments, Castilla crude oil of 15 API was used because it is a type of oil that contains higher amounts of metals mentioned above.

Eighteen (18) reactors were made, one for each experiment. All tubes were hermetically sealed in order to control gas leakage, prevent the entry of oxygen (to prevent oxidation of metals), and control the pressure steadily. Heat was applied to each of the reactors with an infrared lamp intermittently to allow the movement of crude oil through the sediments and come into contact with the metals. The reactors were left under these conditions for 30 days allowing the saturation of all sediments.

Soil samples contained in the reactor were taken at 15, 25 and 30 days. The reactor has 3 windows of 6 cm in diameter in an elliptical shape. A double-walled elliptical tube was inserted through the window of the reactor in order to take a sample of 35 to 40 g. Each sample was stored in petri dishes. The samples were immediately exposed to UV radiation at 366 nm, obtaining the fluorescence between 10 and 40 seconds of being exposed depending on the metal. Nickel emitted fluorescence after 10 seconds, vanadium at 15 or 20 seconds, while iron and copper at 40 and 50 seconds. In samples with a mixture of the 4 metals, fluorescence was observed at 10 seconds, the metal-free samples did not reflect any response. During these tests, it was observed that the intensity depended on metal concentration and sample humidity. With vanadium and nickel, the samples with lowest concentrations had the highest fluorescence.

Additionally, real sediment samples extracted from groundwater impregnated with oil and own sludge from the drilling operation at the well were evaluated. The samples were extracted from the well at 5,000, 6,400 and 7,400 feet deep in order to have a wide range of samples to identify the influence of external agents on the fluorescence reaction. Under UV radiation, it was found that pollutants distort the fluorescence and cause interference in the analysis.

### 2. Development of the Computational Model

The fluorescence emitted by the samples was captured by a camera (Nikon D80 features a 10.2-megapixel CCD sensor, Nikon DX format (23.6×15.8 mm with a 1.5× conversion factor)), which is stored on the hard disk storage (disk or Hard Disk Flask) of the camera to be entered into the model. The image information is converted into numerical values to be integrated into the computational model as inputs.

This is a neural computational model that is designed, trained, validated and tested with data from the fluorescence of images obtained by the camera in order to identify the different levels of fluorescence associated with the presence of oil. All this information comes as inputs to the model. This model can integrate inputs from known and unknown variables, allowing a very high performance during prediction compared with conventional methods, as well as building the standardization curve within a range from 0 to 1. 0, which corresponds to the absence (0) and presence (1) of oil.

Once the image data containing the points of fluorescence have been loaded by the software, each of the pixels of the images are identified are entered into the neural computational system for processing. The neuronal system counts the number of fluorescent spots from the pixels determining the fluorescence level of the sample based on this information.

Fluorescence data generated by metals and interpreted by the computational model (see Table 1 and Table 2) is fed into a library system of image processing that is integrated into the model. The library is responsible for identifying the points (pixels) of fluorescence in the image, which creates an array of data as well as storing the position and fluorescence intensity. These data are used as inputs for training the computational model, and then assess the reliability of the model in relation to the expression of different levels of fluorescence (see FIG. 20).

### 3. Quantification of the Fluorescence in Relation to Presence of Petroleum Oil

Fluorescence was determined based on the concentration of the metals in relation to the presence of petroleum oil. This determination was done by using a dual optical fiber (2-600 μm wide, 2 m long) coated with polyether ketone in order to prevent damage and/or interference caused by chemicals and/or physical components. The optical fiber is integrated to a spectrophotometer USBB2000 plus (Ocean Optics, Florida). The equipment identifies the wavelength or spectrum, specifically produced by each metal, and by the different types of petroleum oil, once they are exposed to the UV light. UV radiation is emitted by a Xenon lamp (220 Hz 220-750 nm, 5500 hours 50 Hz), which sends the waves through the optical fiber. This lamp allows calibration of specific wave lengths in order to produce a specific level of radiation. This is important, since each metal has its own specific radiation and wave length.

Results for each sample were obtained after detection with a UV sensor and subsequent analysis by the software described above. Results were compared with the chemical analysis (digestion) of the samples and to the analysis of metal concentrations by atomic absorption, which correlated very well as shown in the tables and figures.

Field samples were taken from different depths and different distances from each other. Surface field samples did not receive any treatment. Samples were stored in plastic bags to preserve the humidity and in the dark. The samples were then transferred to Petri dishes and exposed to UV light (256 nm) for 10 minutes to produce fluorescence. The fluorescence was recorded using the photo-camera, the spectrophometric device, and the software as described above.

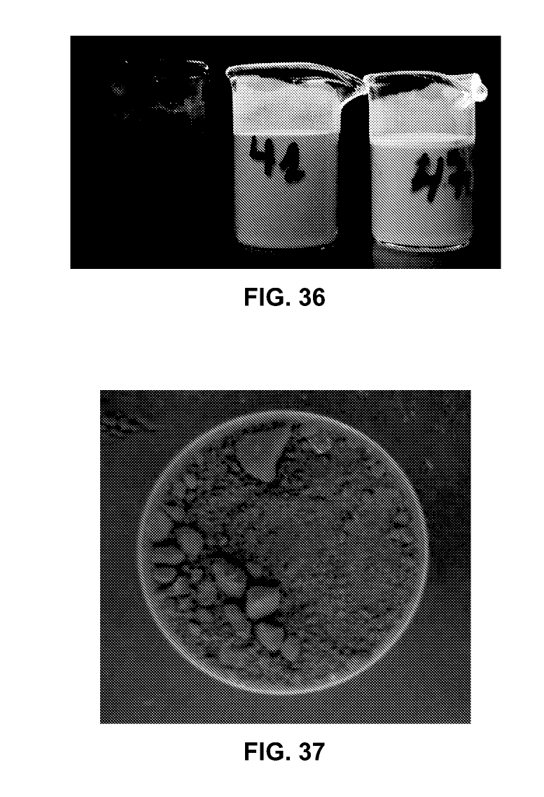

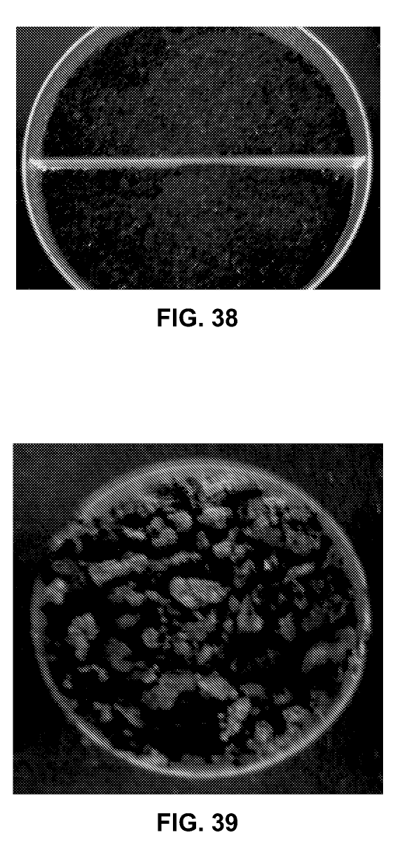

Fluorescence in the sediments was determined by mixing the sediments with petroleum oil of grade 15, 21, 30, 41, 47 API (15 grams). Sediments collected from the field were treated with 0.5 ml of petroleum oil in the Petri dishes in the ratio of 15:0.5. The best results (i.e., highest fluorescence intensity) were observed with samples 41 and 47 API. This was due to the presence of metals, which fluoresce under the UV radiation. Additionally, the concentration of the metals was greater in the higher API samples (FIGS. 35 and 36).

Petroleum oil alone fluoresces according to the grade of API. Several tests were carried out with petroleum oil of different API. Petroleum oil with lower API showed increased fluorescence when it comes in contact with the sediment containing the metals.

### 4. Construction of the Quantitative Computational Modeling:

All information obtained from experiment number two above (i.e., the optical fiber sensor) was used to train the computational model. The samples submitted to the computational model had metals combination at different concentrations. The samples were subjected to different waves intensities in which metals respond with a certain absorption length that indicates metal presence. This procedure was measured by the optical sensor and the results were stored in flat files in the model.

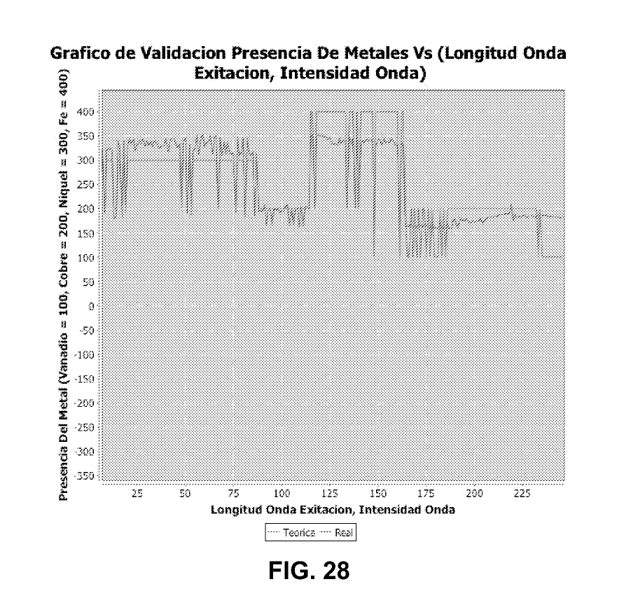

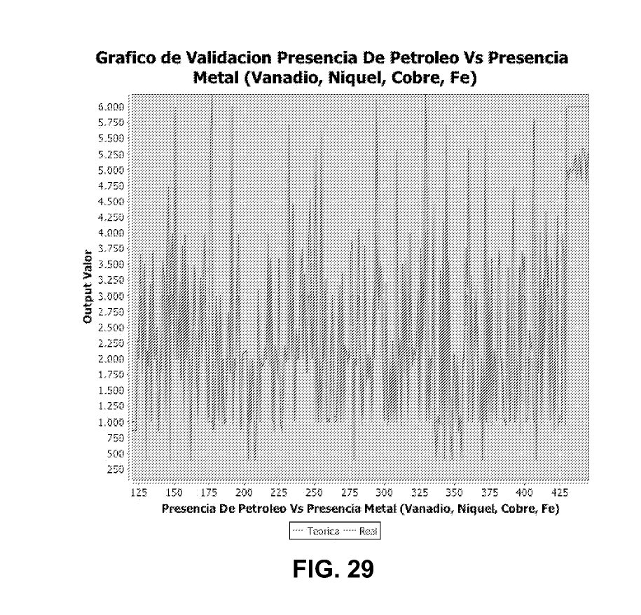

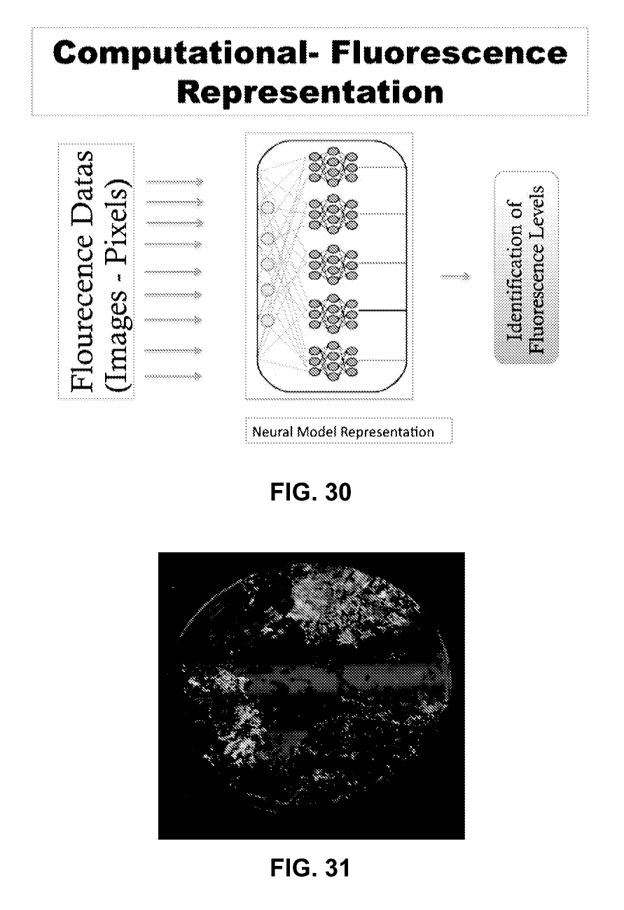

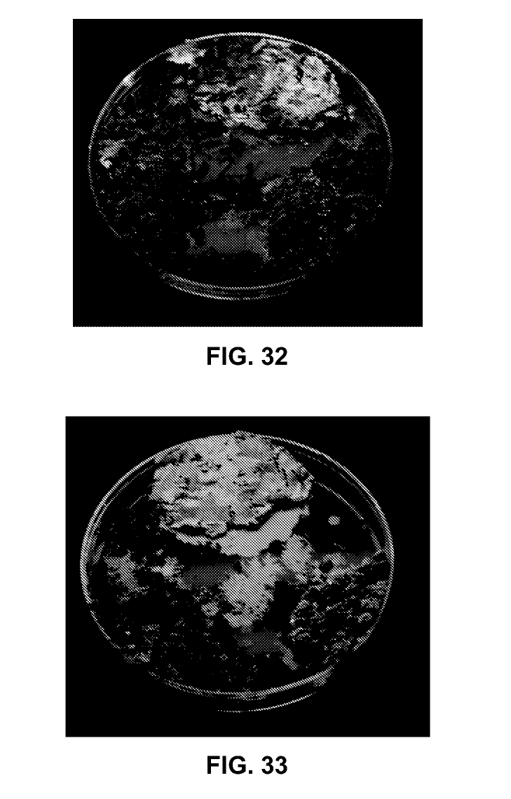

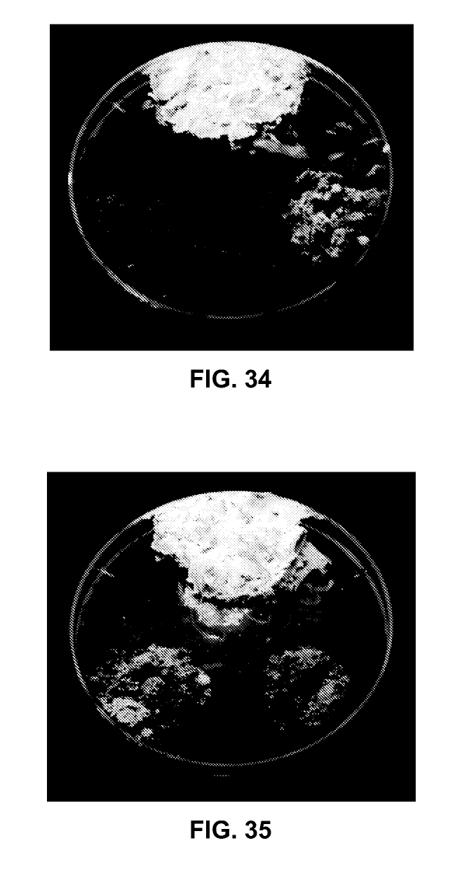

This set of samples was fed to the computational model to determine if the model had the ability to identify which metal was present in a new sample. Metals identification tests such as vanadium, nickel, copper and iron are shown in FIG. 28. Correct identification of the presence or absence of a metal using the computational model was based on the wavelengths at which samples respond, their respective absorption and emission wavelengths, and wave intensity in which samples were irradiated.

With the same set of samples, the model ability to identify metal abundance and the presence or absence of oil was tested (FIGS. 8 and 9 and Table 4). After identifying metal presence, the computational model correctly identified the abundance levels of each identified metal. Based on the above parameters, presence of oil levels were determined.

### 5. Correlation Protocols

Quantitative and qualitative fluorescence data were analyzed and integrated by their own computational model. The combination of the two models produced a final value based on the following equation:

$${\{ f ( x ) = \frac{{e}^{2 x} - 1}{{e}^{2 x} + 1} \}}^{+ b}$$

where (b) is the normalization constant of the data involved in the intersection. Y=f(x) which is the fluorescence of the metals. (x)=concentration of metals.

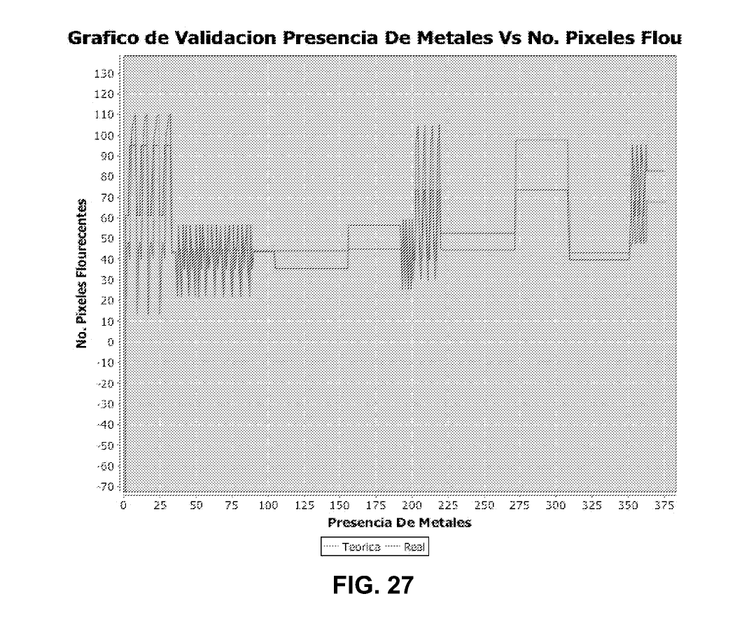

After standardizing the data, a new data array that feeds the new system is constructed. This system contains a new array of connections between data, which represents the relationship between quantitative and qualitative data generating automatically an equivalence table (see Table 3), expressed in digital form to compare fluorescence intensity vs. metal concentration and the presence of oil. The computer model is in charge of giving mathematical values to the qualitative observations, allowing a mathematical integration. This equivalence protocol was demonstrated experimentally on field and in the laboratory (FIG. 27). The system loads as inputs the data from the array determining the corresponding outputs. The array is responsible for integrating the fluorescence intensity with the metals concentration in x, y and z coordinates.

### 6. Quantification and Fluorescence of Metals in Relation to Petroleum API, Using the Computational Model

The fluorescence of the metals was correlated to the concentration of the density of the petroleum, following the protocol of the API (see Table below), based on the relationship between the fluorescence and the grade of the petroleum.

### API Classification.

| Classification of petroleum oil | GRAVITY OF API (° API) |
|---|---|
| Slightly light | >31.1 |
| Medium | 22.3 to 31.1 |
| Heavy | between 10 to 22.3 |
| Extra-heavy | <10 |

Different experiments were performed in order to establish the relationship between the concentration of the metals and the degree or percentage of API. Petroleum samples with API between 10 and 30 required treatment with metals, while petroleum with API higher than 30 did not require any metal treatment, as they were easily detected by the optical sensor.

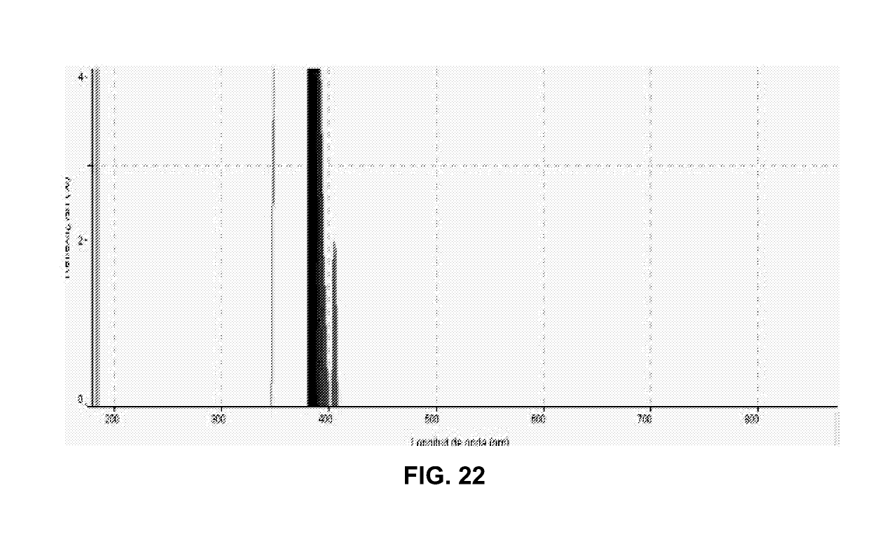

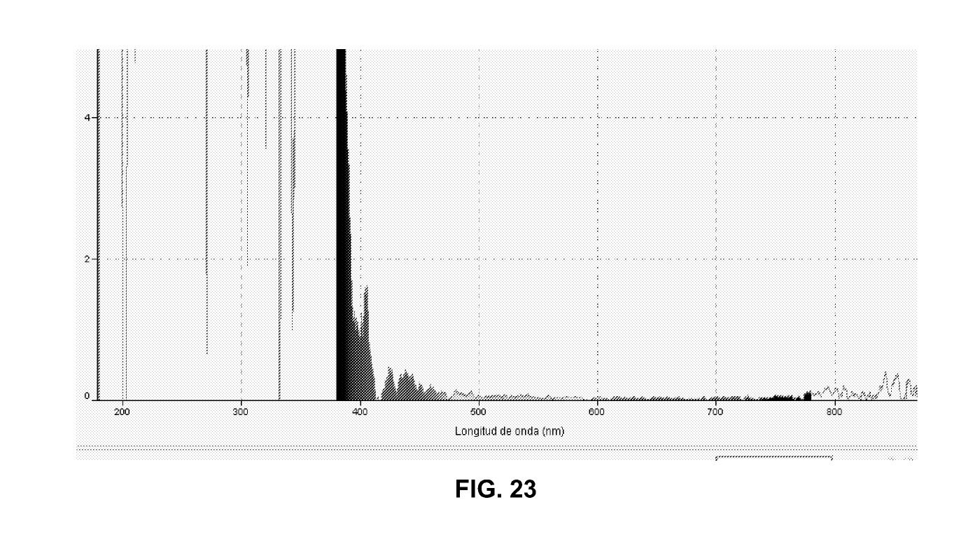

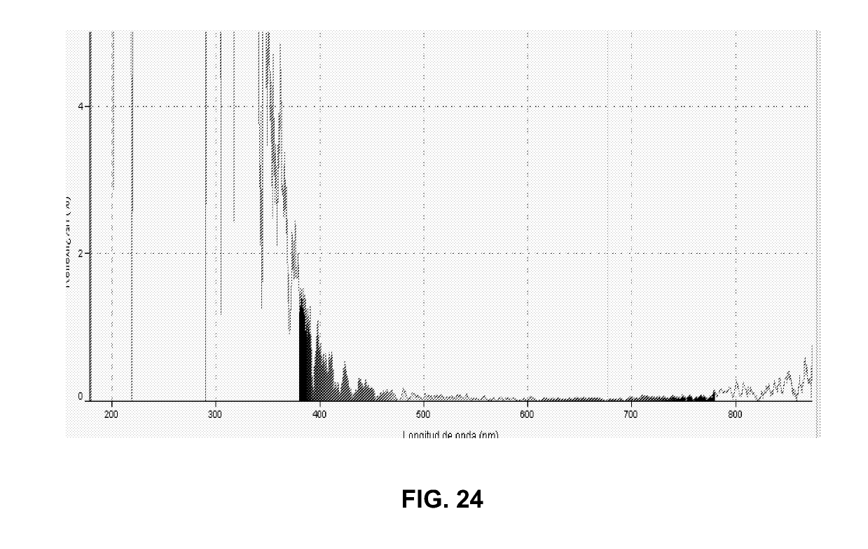

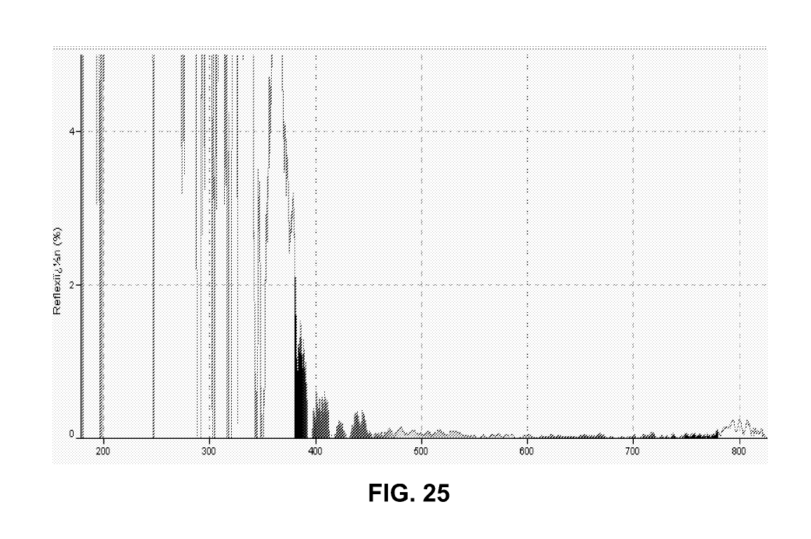

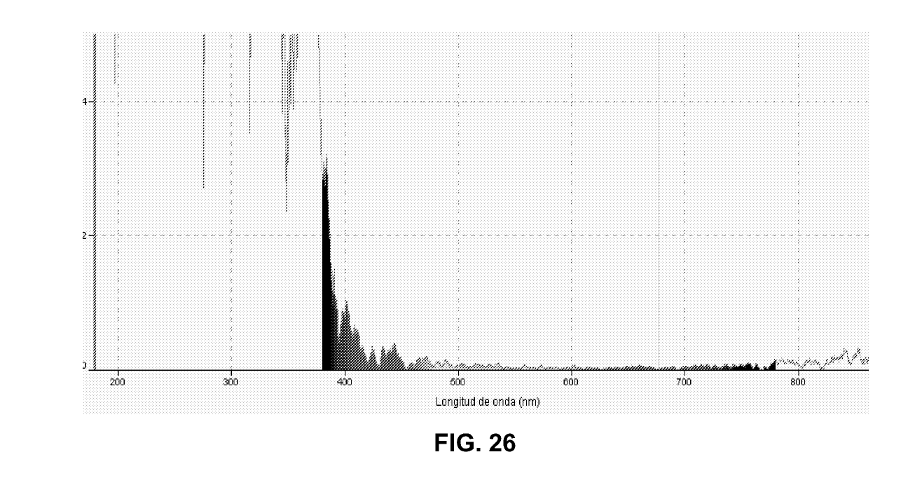

Analysis with optical fiber described above were done, which sends a signal to the spectrometry instrument which expresses a spectrum shown in numeric values. Each of the spectra obtained were used to compare with the values obtained from other theoretical sources, while the numerical values were entered into the analysis software to determine the presence or absence of crude oil in the area. As noted in the experimental results (FIGS. 22-26), measurements were obtained for each of the associated metals with some interference due to physical laboratory conditions. The optimal conditions for reading and adjustment were made in the design of equipment and measurement conditions.

Chemical analysis of different API oil was conducted to determine the incidence on metal concentrations and its different proportions, using the conventional method of atomic absorption. This correlation of variables permits the association table in which according to the reading performed by the spectrometry device to determine if the sample is associated with the presence or absence of oil. Association variables are distributed as follows:

1. Absence of metals associated to crude
2. Presence of a metal at low concentration
3. Presence of two metals
4. Presence of three metals

5. Presence of the 4 metals associated to crude

6. Presence of 4 metals in proportions associated with crude
7. Presence of 4 metals with a clear proportion associated to the API degree.

The partnership model of variables related to the presence or absence of metals is developed in the computational model complementary to the sample reading system, and is essential for the delivery of results. The computational part is responsible for reading the data obtained by spectrometry equipment and of generating conclusions on each of the samples, obtaining an objective assessment of the information to give a precise, measurable and quantifiable answer. Tables 6-9 show the correlation between type of petroleum oil (API gravity) and fluorescence emission.

While the invention has been described with reference to preferred and example embodiments, it will be understood by those skilled in the art that a variety of modifications, additions and deletions are within the scope of the invention, as defined by the following claims.

### TABLES

TABLE 1
Samples with low fluorescence measurements. First column represents
fluorescence level real value. Second column represents the
value measured by the computational model. Values have been
normalized in the range [o. 1] in equation 1.

| Real Value | Result from Model | Absolute Error | Relative error |
|---|---|---|---|
| 0.000000 | 0.000000 | 0.000000 | 0.000000 |
| 0.000000 | 0.000000 | 0.000000 | 0.000000 |
| 0.000000 | 0.000000 | 0.000000 | 0.000000 |
| 0.175000 | 0.175044 | 0.000044 | 0.000251 |
| 0.037500 | 0.038521 | 0.001021 | 0.027227 |
| 0.025000 | 0.025234 | 0.000234 | 0.009360 |
| 0.500000 | 0.500671 | 0.000671 | 0.001342 |
| 0.000000 | 0.000000 | 0.000000 | 0.000000 |
| | | Average Error | 0.004772512 |

TABLE 2
Samples with high fluorescence measurements. First column
represents fluorescence level real value. Second column represents
the value measured by the computational model. Values have
been normalized in the range [o. 1] in equation 1.

| | | | | | Flourescence |
|---|---|---|---|---|---|
| vanadium | copper | nickel | iron | Concentration | (Pixels) |
| 100 | 100 | 100 | 200 | 100 | 13 |
| 100 | 100 | 100 | 200 | 100 | 45 |
| 100 | 100 | 100 | 200 | 100 | 48 |
| 100 | 100 | 200 | 100 | 100 | 40 |
| 100 | 100 | 200 | 100 | 100 | 99 |
| 100 | 100 | 200 | 100 | 100 | 105 |
| 100 | 100 | 200 | 100 | 100 | 109 |
| 100 | 100 | 200 | 100 | 100 | 110 |
| 100 | 100 | 100 | 200 | 100 | 13 |
| 100 | 100 | 100 | 200 | 100 | 45 |
| 100 | 100 | 100 | 200 | 100 | 48 |
| 100 | 100 | 200 | 100 | 100 | 40 |
| 100 | 100 | 200 | 100 | 100 | 99 |
| 100 | 100 | 200 | 100 | 100 | 105 |
| 100 | 100 | 200 | 100 | 100 | 109 |
| 100 | 100 | 200 | 100 | 100 | 110 |
| 100 | 100 | 100 | 200 | 100 | 13 |
| 100 | 100 | 100 | 200 | 100 | 45 |
| 100 | 100 | 100 | 200 | 100 | 48 |
| 100 | 100 | 200 | 100 | 100 | 40 |
| 100 | 100 | 200 | 100 | 100 | 99 |
| 100 | 100 | 200 | 100 | 100 | 105 |
| 100 | 100 | 200 | 100 | 100 | 109 |
| 100 | 100 | 200 | 100 | 100 | 110 |
| 100 | 100 | 100 | 200 | 100 | 13 |
| 100 | 100 | 100 | 200 | 100 | 45 |
| 100 | 100 | 100 | 200 | 100 | 48 |
| 100 | 100 | 200 | 100 | 100 | 40 |
| 100 | 100 | 200 | 100 | 100 | 99 |
| 100 | 100 | 200 | 100 | 100 | 105 |
| 100 | 100 | 200 | 100 | 100 | 109 |
| 100 | 100 | 200 | 100 | 100 | 110 |
| 200 | 100 | 100 | 100 | 8.8 | 42.6 |
| 200 | 100 | 100 | 100 | 8.8 | 42.6 |
| 200 | 100 | 100 | 100 | 8.8 | 42.6 |
| 200 | 100 | 100 | 100 | 8.8 | 42.6 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 100 | 100 | 200 | 100 | 10 | 43.95906613 |
| 100 | 200 | 100 | 100 | 10 | 44.95802835 |
| 100 | 100 | 100 | 200 | 10 | 23 |
| 100 | 100 | 200 | 100 | 10 | 43.95906613 |
| 100 | 200 | 100 | 100 | 10 | 44.95802835 |
| 200 | 100 | 100 | 100 | 10 | 45 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 200 | 100 | 100 | 100 | 10 | 43.8370079 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 200 | 100 | 100 | 10 | 44.15336165 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 200 | 100 | 10 | 44.82479244 |
| 100 | 100 | 100 | 200 | 16.66 | 43 |
| 100 | 100 | 200 | 100 | 16.66 | 43 |
| 100 | 100 | 100 | 200 | 16.66 | 43 |
| 100 | 100 | 200 | 100 | 16.66 | 43 |
| 100 | 100 | 100 | 200 | 16.66 | 43 |
| 100 | 100 | 200 | 100 | 16.66 | 43 |
| 100 | 100 | 100 | 200 | 16.66 | 43 |
| 100 | 100 | 200 | 100 | 16.66 | 43 |
| 100 | 100 | 100 | 200 | 50 | 30 |
| 100 | 100 | 100 | 200 | 50 | 54.60804676 |
| 100 | 100 | 200 | 100 | 50 | 96.16505767 |
| 100 | 100 | 200 | 100 | 50 | 105 |
| 100 | 200 | 100 | 100 | 50 | 45.03965521 |
| 100 | 100 | 100 | 200 | 50 | 30 |
| 100 | 100 | 100 | 200 | 50 | 54.60804676 |
| 100 | 100 | 200 | 100 | 50 | 96.16505767 |
| 100 | 100 | 200 | 100 | 50 | 105 |
| 100 | 200 | 100 | 100 | 50 | 45.03965521 |
| 100 | 100 | 100 | 200 | 50 | 30 |
| 100 | 100 | 100 | 200 | 50 | 54.60804676 |
| 100 | 100 | 200 | 100 | 50 | 96.16505767 |
| 100 | 100 | 200 | 100 | 50 | 105 |
| 100 | 200 | 100 | 100 | 50 | 45.03965521 |
| 100 | 100 | 100 | 200 | 50 | 30 |
| 100 | 100 | 100 | 200 | 50 | 54.60804676 |
| 100 | 100 | 200 | 100 | 50 | 96.16505767 |
| 100 | 100 | 200 | 100 | 50 | 105 |
| 100 | 200 | 100 | 100 | 50 | 45.03965521 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 200 | 100 | 100 | 50 | 44.35380607 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 200 | 100 | 50 | 97.72042636 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 50 | 43.27621815 |
| 100 | 100 | 100 | 200 | 100 | 48 |
| 100 | 100 | 200 | 100 | 100 | 48 |
| 200 | 100 | 100 | 100 | 100 | 67.98302361 |
| 100 | 100 | 100 | 200 | 100 | 48 |
| 100 | 100 | 200 | 100 | 100 | 48 |
| 200 | 100 | 100 | 100 | 100 | 67.98302361 |
| 100 | 100 | 100 | 200 | 100 | 48 |
| 100 | 100 | 200 | 100 | 100 | 48 |
| 200 | 100 | 100 | 100 | 100 | 67.98302361 |
| 100 | 100 | 100 | 200 | 100 | 48 |
| 100 | 100 | 200 | 100 | 100 | 48 |
| 200 | 100 | 100 | 100 | 100 | 67.98302361 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |
| 200 | 100 | 100 | 100 | 100 | 67.56639466 |

TABLE 3
Equivalance between qualitative data and quantitative data including
concentration and fluoresence levels of the metals

| | | | LO | | Petroleum |
|---|---|---|---|---|---|
| LO Va | IO Va | LO Cu | Fe | PPM | Abundance |
| 410 | 5 | 475 | 3 | 50 | 4 |
| 352.6 | 4.3 | 408.5 | 2.58 | 43 | 3.44 |
| 401.8 | 4.9 | 465.5 | 2.94 | 49 | 3.92 |
| 196.8 | 2.4 | 228 | 1.44 | 24 | 1.92 |
| 369 | 4.5 | 427.5 | 2.7 | 45 | 3.6 |
| 90.2 | 1.1 | 104.5 | 0.66 | 11 | 0.88 |
| 164 | 2 | 190 | 1.2 | 20 | 1.6 |
| 352.6 | 4.3 | 408.5 | 2.58 | 43 | 3.44 |
| 213.2 | 2.6 | 247 | 1.56 | 26 | 2.08 |
| 410 | 5 | 475 | 3 | 15 | 4 |
| 27.33333333 | 0.333333333 | 9.5 | 0.06 | 1 | 0.196666667 |
| 54.66666667 | 0.666666667 | 19 | 0.12 | 2 | 0.393333333 |
| 1312 | 16 | 456 | 2.88 | 48 | 9.44 |
| 1284.666667 | 15.66666667 | 446.5 | 2.82 | 47 | 9.243333333 |
| 683.3333333 | 8.333333333 | 237.5 | 1.5 | 25 | 4.916666667 |
| 792.6666667 | 9.666666667 | 275.5 | 1.74 | 29 | 5.703333333 |
| 574 | 7 | 199.5 | 1.26 | 21 | 4.13 |
| 164 | 2 | 57 | 0.36 | 6 | 1.18 |
| 929.3333333 | 11.33333333 | 323 | 2.04 | 34 | 6.686666667 |
| 109.3333333 | 1.333333333 | 38 | 0.24 | 4 | 0.786666667 |
| 519.3333333 | 6.333333333 | 180.5 | 1.14 | 19 | 3.736666667 |
| 492 | 6 | 171 | 1.08 | 18 | 3.54 |
| 847.3333333 | 10.33333333 | 294.5 | 1.86 | 31 | 6.096666667 |
| 218.6666667 | 2.666666667 | 76 | 0.48 | 8 | 1.573333333 |
| 574 | 7 | 199.5 | 1.26 | 21 | 4.13 |
| 1148 | 14 | 399 | 2.52 | 42 | 8.26 |
| 765.3333333 | 9.333333333 | 266 | 1.68 | 28 | 5.506666667 |
| 164 | 2 | 57 | 0.36 | 6 | 1.18 |
| 464.6666667 | 5.666666667 | 161.5 | 1.02 | 17 | 3.343333333 |
| 191.3333333 | 2.333333333 | 66.5 | 0.42 | 7 | 1.376666667 |
| 1366.666667 | 16.66666667 | 475 | 3 | 50 | 9.833333333 |
| 628.6666667 | 7.666666667 | 218.5 | 1.38 | 23 | 4.523333333 |
| 437.3333333 | 5.333333333 | 152 | 0.96 | 16 | 3.146666667 |
| 1038.666667 | 12.66666667 | 361 | 2.28 | 38 | 7.473333333 |
| 1366.666667 | 16.66666667 | 475 | 3 | 50 | 9.833333333 |
| 1339.333333 | 16.33333333 | 465.5 | 2.94 | 49 | 9.636666667 |
| 1230 | 15 | 427.5 | 2.7 | 45 | 8.85 |
| 738 | 9 | 256.5 | 1.62 | 27 | 5.31 |
| 820 | 10 | 285 | 1.8 | 30 | 5.9 |
| 191.3333333 | 2.333333333 | 66.5 | 0.42 | 7 | 1.376666667 |
| 191.3333333 | 2.333333333 | 66.5 | 0.42 | 7 | 1.376666667 |
| 218.6666667 | 2.666666667 | 76 | 0.48 | 8 | 1.573333333 |
| 300.6666667 | 3.666666667 | 104.5 | 0.66 | 11 | 2.163333333 |
| 519.3333333 | 6.333333333 | 180.5 | 1.14 | 19 | 3.736666667 |
| 1093.333333 | 13.33333333 | 380 | 2.4 | 40 | 7.866666667 |
| 902 | 11 | 313.5 | 1.98 | 33 | 6.49 |
| 382.6666667 | 4.666666667 | 133 | 0.84 | 14 | 2.753333333 |
| 820 | 10 | 285 | 1.8 | 30 | 5.9 |
| 929.3333333 | 11.33333333 | 323 | 2.04 | 34 | 6.686666667 |
| 1339.333333 | 16.33333333 | 465.5 | 2.94 | 49 | 9.636666667 |
| 1284.666667 | 15.66666667 | 446.5 | 2.82 | 47 | 9.243333333 |
| 475 | 20 | 0 | 0 | 50 | 10 |
| 475 | 20 | 0 | 0 | 50 | 10 |
| 389.5 | 16.4 | 0 | 0 | 41 | 8.2 |
| 247 | 10.4 | 0 | 0 | 26 | 5.2 |
| 133 | 5.6 | 0 | 0 | 14 | 2.8 |
| 256.5 | 10.8 | 0 | 0 | 27 | 5.4 |
| 418 | 17.6 | 0 | 0 | 44 | 8.8 |
| 95 | 4 | 0 | 0 | 10 | 2 |
| 380 | 16 | 0 | 0 | 40 | 8 |
| 323 | 13.6 | 0 | 0 | 34 | 6.8 |
| 95 | 4 | 0 | 0 | 10 | 2 |
| 446.5 | 18.8 | 0 | 0 | 47 | 9.4 |
| 275.5 | 11.6 | 0 | 0 | 29 | 5.8 |
| 323 | 13.6 | 0 | 0 | 34 | 6.8 |
| 446.5 | 18.8 | 0 | 0 | 47 | 9.4 |
| 266 | 11.2 | 0 | 0 | 28 | 5.6 |
| 209 | 8.8 | 0 | 0 | 22 | 4.4 |
| 342 | 14.4 | 0 | 0 | 36 | 7.2 |
| 342 | 14.4 | 0 | 0 | 36 | 7.2 |
| 228 | 9.6 | 0 | 0 | 24 | 4.8 |
| 351.5 | 14.8 | 0 | 0 | 37 | 7.4 |
| 180.5 | 7.6 | 0 | 0 | 19 | 3.8 |
| 437 | 18.4 | 0 | 0 | 46 | 9.2 |
| 370.5 | 15.6 | 0 | 0 | 39 | 7.8 |
| 152 | 6.4 | 0 | 0 | 16 | 3.2 |
| 104.5 | 4.4 | 0 | 0 | 11 | 2.2 |
| 408.5 | 17.2 | 0 | 0 | 43 | 8.6 |
| 456 | 19.2 | 0 | 0 | 48 | 9.6 |
| 351.5 | 14.8 | 0 | 0 | 37 | 7.4 |
| 152 | 6.4 | 0 | 0 | 16 | 3.2 |
| 475 | 20 | 0 | 0 | 50 | 10 |
| 285 | 12 | 0 | 0 | 30 | 6 |
| 85.5 | 3.6 | 0 | 0 | 9 | 1.8 |
| 180.5 | 7.6 | 0 | 0 | 19 | 3.8 |
| 351.5 | 14.8 | 0 | 0 | 37 | 7.4 |
| 275.5 | 11.6 | 0 | 0 | 29 | 5.8 |
| 180.5 | 7.6 | 0 | 0 | 19 | 3.8 |
| 152 | 6.4 | 0 | 0 | 16 | 3.2 |
| 247 | 10.4 | 0 | 0 | 26 | 5.2 |
| 389.5 | 16.4 | 0 | 0 | 41 | 8.2 |
| 266 | 11.2 | 0 | 0 | 28 | 5.6 |
| 171 | 7.2 | 0 | 0 | 18 | 3.6 |
| 437 | 18.4 | 0 | 0 | 46 | 9.2 |
| 47.5 | 2 | 0 | 0 | 5 | 1 |
| 142.5 | 6 | 0 | 0 | 15 | 3 |
| 66.5 | 2.8 | 0 | 0 | 7 | 1.4 |
| 465.5 | 19.6 | 0 | 0 | 49 | 9.8 |
| 104.5 | 4.4 | 0 | 0 | 11 | 2.2 |
| 304 | 12.8 | 0 | 0 | 32 | 6.4 |
| 85.5 | 3.6 | 0 | 0 | 9 | 1.8 |
| 47.5 | 2 | 0 | 0 | 5 | 1 |
| 475 | 20 | 0 | 0 | 50 | 10 |
| 475 | 20 | 0 | 0 | 50 | 10 |
| 389.5 | 16.4 | 0 | 0 | 41 | 8.2 |
| 247 | 10.4 | 0 | 0 | 26 | 5.2 |
| 133 | 5.6 | 0 | 0 | 14 | 2.8 |
| 256.5 | 10.8 | 0 | 0 | 27 | 5.4 |
| 418 | 17.6 | 0 | 0 | 44 | 8.8 |
| 95 | 4 | 0 | 0 | 10 | 2 |
| 380 | 16 | 0 | 0 | 40 | 8 |
| 323 | 13.6 | 0 | 0 | 34 | 6.8 |
| 95 | 4 | 0 | 0 | 10 | 2 |
| 446.5 | 18.8 | 0 | 0 | 47 | 9.4 |
| 275.5 | 11.6 | 0 | 0 | 29 | 5.8 |
| 323 | 13.6 | 0 | 0 | 34 | 6.8 |
| 446.5 | 18.8 | 0 | 0 | 47 | 9.4 |
| 266 | 11.2 | 0 | 0 | 28 | 5.6 |
| 209 | 8.8 | 0 | 0 | 22 | 4.4 |
| 342 | 14.4 | 0 | 0 | 36 | 7.2 |
| 342 | 14.4 | 0 | 0 | 36 | 7.2 |
| 228 | 9.6 | 0 | 0 | 24 | 4.8 |
| 351.5 | 14.8 | 0 | 0 | 37 | 7.4 |
| 180.5 | 7.6 | 0 | 0 | 19 | 3.8 |
| 437 | 18.4 | 0 | 0 | 46 | 9.2 |
| 370.5 | 15.6 | 0 | 0 | 39 | 7.8 |
| 152 | 6.4 | 0 | 0 | 16 | 3.2 |
| 104.5 | 4.4 | 0 | 0 | 11 | 2.2 |
| 408.5 | 17.2 | 0 | 0 | 43 | 8.6 |
| 456 | 19.2 | 0 | 0 | 48 | 9.6 |
| 351.5 | 14.8 | 0 | 0 | 37 | 7.4 |
| 152 | 6.4 | 0 | 0 | 16 | 3.2 |
| 475 | 20 | 0 | 0 | 50 | 10 |
| 285 | 12 | 0 | 0 | 30 | 6 |
| 85.5 | 3.6 | 0 | 0 | 9 | 1.8 |
| 180.5 | 7.6 | 0 | 0 | 19 | 3.8 |
| 351.5 | 14.8 | 0 | 0 | 37 | 7.4 |
| 275.5 | 11.6 | 0 | 0 | 29 | 5.8 |
| 180.5 | 7.6 | 0 | 0 | 19 | 3.8 |
| 152 | 6.4 | 0 | 0 | 16 | 3.2 |
| 247 | 10.4 | 0 | 0 | 26 | 5.2 |
| 389.5 | 16.4 | 0 | 0 | 41 | 8.2 |
| 266 | 11.2 | 0 | 0 | 28 | 5.6 |
| 171 | 7.2 | 0 | 0 | 18 | 3.6 |
| 437 | 18.4 | 0 | 0 | 46 | 9.2 |
| 47.5 | 2 | 0 | 0 | 5 | 1 |
| 142.5 | 6 | 0 | 0 | 15 | 3 |
| 66.5 | 2.8 | 0 | 0 | 7 | 1.4 |
| 465.5 | 19.6 | 0 | 0 | 49 | 9.8 |
| 104.5 | 4.4 | 0 | 0 | 11 | 2.2 |
| 304 | 12.8 | 0 | 0 | 32 | 6.4 |
| 85.5 | 3.6 | 0 | 0 | 9 | 1.8 |
| 47.5 | 2 | 0 | 0 | 5 | 1 |
| 0 | 0 | 423 | 17 | 50 | 8.5 |
| 0 | 0 | 423 | 17 | 50 | 8.5 |
| 0 | 0 | 270.72 | 10.88 | 49 | 5.44 |
| 0 | 0 | 50.76 | 2.04 | 29 | 1.02 |
| 0 | 0 | 186.12 | 7.48 | 4 | 3.74 |
| 0 | 0 | 194.58 | 7.82 | 50 | 3.91 |
| 0 | 0 | 236.88 | 9.52 | 30 | 4.76 |
| 0 | 0 | 304.56 | 12.24 | 6 | 6.12 |
| 0 | 0 | 67.68 | 2.72 | 8 | 1.36 |
| 0 | 0 | 423 | 17 | 10 | 8.5 |
| 0 | 0 | 143.82 | 5.78 | 24 | 2.89 |
| 0 | 0 | 169.2 | 6.8 | 48 | 3.4 |
| 0 | 0 | 101.52 | 4.08 | 48 | 2.04 |
| 0 | 0 | 329.94 | 13.26 | 21 | 6.63 |
| 0 | 0 | 59.22 | 2.38 | 35 | 1.19 |
| 0 | 0 | 414.54 | 16.66 | 11 | 8.33 |
| 0 | 0 | 50.76 | 2.04 | 4 | 1.02 |
| 0 | 0 | 262.26 | 10.54 | 37 | 5.27 |
| 0 | 0 | 253.8 | 10.2 | 27 | 5.1 |
| 0 | 0 | 203.04 | 8.16 | 26 | 4.08 |
| 0 | 0 | 169.2 | 6.8 | 37 | 3.4 |
| 0 | 0 | 219.96 | 8.84 | 47 | 4.42 |
| 0 | 0 | 414.54 | 16.66 | 31 | 8.33 |
| 0 | 0 | 194.58 | 7.82 | 48 | 3.91 |
| 0 | 0 | 287.64 | 11.56 | 20 | 5.78 |
| 0 | 0 | 135.36 | 5.44 | 29 | 2.72 |
| 0 | 0 | 59.22 | 2.38 | 1 | 1.19 |
| 0 | 0 | 42.3 | 1.7 | 3 | 0.85 |
| 0 | 0 | 414.54 | 16.66 | 50 | 8.33 |
| 0 | 0 | 236.88 | 9.52 | 41 | 4.76 |
| 0 | 0 | 101.52 | 4.08 | 5 | 2.04 |
| 0 | 0 | 304.56 | 12.24 | 35 | 6.12 |
| 0 | 0 | 211.5 | 8.5 | 26 | 4.25 |
| 0 | 0 | 177.66 | 7.14 | 33 | 3.57 |
| 0 | 0 | 33.84 | 1.36 | 16 | 0.68 |
| 0 | 0 | 304.56 | 12.24 | 37 | 6.12 |
| 0 | 0 | 372.24 | 14.96 | 12 | 7.48 |
| 0 | 0 | 423 | 17 | 33 | 8.5 |
| 0 | 0 | 16.92 | 0.68 | 21 | 0.34 |
| 0 | 0 | 76.14 | 3.06 | 14 | 1.53 |
| 0 | 0 | 33.84 | 1.36 | 50 | 0.68 |
| 0 | 0 | 313.02 | 12.58 | 35 | 6.29 |
| 0 | 0 | 363.78 | 14.62 | 11 | 7.31 |
| 0 | 0 | 346.86 | 13.94 | 4 | 6.97 |
| 0 | 0 | 355.32 | 14.28 | 39 | 7.14 |
| 0 | 0 | 304.56 | 12.24 | 13 | 6.12 |
| 0 | 0 | 169.2 | 6.8 | 44 | 3.4 |
| 0 | 0 | 329.94 | 13.26 | 3 | 6.63 |
| 0 | 0 | 16.92 | 0.68 | 10 | 0.34 |

TABLE 4
Metal presence and abundance levels. Training data table.
The results were amplified to thousands for a better view
of the results in the computational model.

| | | | API | |
|---|---|---|---|---|
| Excitement length | Wave length | Wave intensity | degree | Metal type |
| 232 | 501.2 | 0.012 | 41 | 300 |
| 232 | 502.24 | 0.013 | 41 | 300 |
| 232 | 503.63 | 0.014 | 41 | 300 |
| 324.8 | 411.78 | 0.017 | 15 | 200 |
| 232 | 502.94 | 0.021 | 41 | 300 |
| 232 | 502.59 | 0.022 | 41 | 300 |
| 232 | 501.89 | 0.023 | 41 | 300 |
| 324.8 | 417.87 | 0.024 | 15 | 200 |
| 232 | 501.55 | 0.025 | 41 | 300 |
| 232 | 503.29 | 0.025 | 41 | 300 |
| 232 | 503.98 | 0.026 | 41 | 300 |
| 232 | 500.85 | 0.027 | 41 | 300 |
| 324.8 | 411.06 | 0.035 | 15 | 200 |
| 324.8 | 415.36 | 0.038 | 15 | 200 |
| 232 | 501.2 | 0.038 | 15 | 300 |
| 232 | 504.33 | 0.038 | 41 | 300 |
| 324.8 | 411.42 | 0.039 | 15 | 200 |
| 232 | 501.55 | 0.039 | 15 | 300 |
| 324.8 | 418.23 | 0.041 | 15 | 200 |
| 232 | 495.62 | 0.042 | 15 | 300 |
| 232 | 502.59 | 0.042 | 15 | 300 |
| 232 | 497.02 | 0.044 | 15 | 300 |
| 232 | 502.24 | 0.044 | 15 | 300 |
| 232 | 500.5 | 0.044 | 41 | 300 |
| 232 | 495.27 | 0.045 | 15 | 300 |
| 232 | 501.89 | 0.046 | 15 | 300 |
| 232 | 497.37 | 0.047 | 15 | 300 |
| 232 | 502.94 | 0.047 | 15 | 300 |
| 232 | 506.76 | 0.047 | 41 | 300 |
| 232 | 500.85 | 0.048 | 15 | 300 |
| 232 | 507.81 | 0.048 | 41 | 300 |
| 232 | 500.15 | 0.049 | 15 | 300 |
| 232 | 506.42 | 0.049 | 41 | 300 |
| 232 | 500.5 | 0.05 | 15 | 300 |
| 232 | 496.67 | 0.051 | 15 | 300 |
| 232 | 505.03 | 0.051 | 41 | 300 |
| 232 | 505.37 | 0.052 | 41 | 300 |
| 232 | 507.11 | 0.052 | 41 | 300 |
| 232 | 507.46 | 0.052 | 41 | 300 |
| 232 | 503.29 | 0.054 | 15 | 300 |
| 232 | 500.15 | 0.054 | 41 | 300 |
| 232 | 499.81 | 0.055 | 15 | 300 |
| 232 | 503.98 | 0.055 | 15 | 300 |
| 232 | 503.63 | 0.056 | 15 | 300 |
| 232 | 497.71 | 0.057 | 15 | 300 |
| 232 | 504.68 | 0.058 | 41 | 300 |
| 232 | 495.97 | 0.059 | 15 | 300 |
| 324.8 | 428.23 | 0.06 | 15 | 200 |
| 232 | 498.06 | 0.06 | 15 | 300 |
| 232 | 504.33 | 0.06 | 15 | 300 |
| 324.8 | 418.58 | 0.061 | 15 | 200 |
| 232 | 496.32 | 0.061 | 15 | 300 |
| 324.8 | 414.65 | 0.062 | 15 | 200 |
| 232 | 499.81 | 0.062 | 41 | 300 |
| 232 | 505.72 | 0.062 | 41 | 300 |
| 232 | 506.42 | 0.063 | 15 | 300 |
| 232 | 497.02 | 0.063 | 41 | 300 |
| 232 | 507.11 | 0.064 | 15 | 300 |
| 232 | 507.46 | 0.064 | 15 | 300 |
| 232 | 506.07 | 0.064 | 41 | 300 |
| 232 | 498.76 | 0.066 | 15 | 300 |
| 232 | 499.11 | 0.068 | 15 | 300 |
| 232 | 506.76 | 0.068 | 15 | 300 |
| 232 | 497.37 | 0.068 | 41 | 300 |
| 232 | 506.07 | 0.07 | 15 | 300 |
| 232 | 507.81 | 0.07 | 15 | 300 |
| 324.8 | 428.59 | 0.073 | 15 | 200 |
| 232 | 504.68 | 0.074 | 15 | 300 |
| 232 | 495.97 | 0.074 | 41 | 300 |
| 232 | 498.41 | 0.075 | 15 | 300 |
| 232 | 496.67 | 0.075 | 41 | 300 |
| 232 | 499.46 | 0.076 | 15 | 300 |
| 232 | 496.32 | 0.076 | 41 | 300 |
| 232 | 497.71 | 0.077 | 41 | 300 |
| 232 | 498.06 | 0.079 | 41 | 300 |
| 324.8 | 418.94 | 0.08 | 15 | 200 |
| 232 | 505.72 | 0.081 | 15 | 300 |
| 232 | 495.62 | 0.081 | 41 | 300 |
| 232 | 499.46 | 0.081 | 41 | 300 |
| 232 | 505.03 | 0.082 | 15 | 300 |
| 324.8 | 414.29 | 0.083 | 15 | 200 |
| 232 | 505.37 | 0.084 | 15 | 300 |
| 232 | 499.11 | 0.085 | 41 | 300 |
| 232 | 495.27 | 0.086 | 41 | 300 |
| 232 | 498.76 | 0.087 | 41 | 300 |
| 324.8 | 415 | 0.096 | 15 | 200 |
| 232 | 498.41 | 0.097 | 41 | 300 |
| 324.8 | 420.02 | 0.099 | 15 | 200 |
| 324.8 | 421.8 | 0.108 | 15 | 200 |
| 324.8 | 419.3 | 0.111 | 15 | 200 |
| 324.8 | 427.87 | 0.116 | 15 | 200 |
| 324.8 | 415.36 | 0.119 | 41 | 200 |
| 324.8 | 421.45 | 0.121 | 15 | 200 |
| 324.8 | 422.16 | 0.121 | 15 | 200 |
| 324.8 | 415.72 | 0.128 | 41 | 200 |
| 324.8 | 422.88 | 0.13 | 15 | 200 |
| 324.8 | 422.52 | 0.143 | 15 | 200 |
| 324.8 | 423.59 | 0.147 | 15 | 200 |
| 324.8 | 419.66 | 0.152 | 15 | 200 |
| 324.8 | 420.73 | 0.165 | 15 | 200 |
| 324.8 | 420.37 | 0.168 | 15 | 200 |
| 324.8 | 427.16 | 0.17 | 15 | 200 |
| 324.8 | 427.52 | 0.172 | 15 | 200 |
| 324.8 | 415 | 0.173 | 41 | 200 |
| 324.8 | 423.23 | 0.18 | 15 | 200 |
| 324.8 | 416.08 | 0.181 | 41 | 200 |
| 324.8 | 421.09 | 0.191 | 15 | 200 |
| 324.8 | 425.02 | 0.211 | 15 | 200 |
| 324.8 | 414.65 | 0.223 | 41 | 200 |
| 324.8 | 426.45 | 0.238 | 15 | 200 |
| 324.8 | 414.29 | 0.247 | 41 | 200 |
| 324.8 | 424.66 | 0.251 | 15 | 200 |
| 324.8 | 426.8 | 0.254 | 15 | 200 |
| 324.8 | 423.95 | 0.261 | 15 | 200 |
| 248.3 | 508.85 | 0.265 | 15 | 400 |
| 248.3 | 509.54 | 0.266 | 15 | 400 |
| 324.8 | 426.09 | 0.267 | 15 | 200 |
| 248.3 | 509.19 | 0.269 | 15 | 400 |
| 248.3 | 509.89 | 0.276 | 15 | 400 |
| 248.3 | 508.5 | 0.277 | 15 | 400 |
| 248.3 | 508.15 | 0.283 | 15 | 400 |
| 248.3 | 507.46 | 0.284 | 15 | 400 |
| 248.3 | 507.81 | 0.287 | 15 | 400 |
| 248.3 | 503.29 | 0.289 | 15 | 400 |
| 248.3 | 506.42 | 0.29 | 15 | 400 |
| 248.3 | 498.06 | 0.293 | 15 | 400 |
| 248.3 | 498.76 | 0.293 | 15 | 400 |
| 248.3 | 497.71 | 0.295 | 15 | 400 |
| 248.3 | 497.02 | 0.302 | 15 | 400 |
| 248.3 | 506.76 | 0.302 | 15 | 400 |
| 248.3 | 499.11 | 0.303 | 15 | 400 |
| 248.3 | 502.94 | 0.303 | 15 | 400 |
| 248.3 | 500.5 | 0.304 | 15 | 400 |
| 324.8 | 424.3 | 0.306 | 15 | 200 |
| 248.3 | 507.11 | 0.306 | 15 | 400 |
| 248.3 | 498.41 | 0.308 | 15 | 400 |
| 248.3 | 505.37 | 0.308 | 15 | 400 |
| 324.8 | 425.73 | 0.31 | 15 | 200 |
| 248.3 | 506.07 | 0.312 | 15 | 400 |
| 324.8 | 425.38 | 0.313 | 15 | 200 |
| 248.3 | 497.37 | 0.314 | 15 | 400 |
| 248.3 | 502.24 | 0.314 | 15 | 400 |
| 248.3 | 500.15 | 0.316 | 15 | 400 |
| 248.3 | 504.68 | 0.317 | 15 | 400 |
| 248.3 | 502.59 | 0.318 | 15 | 400 |
| 248.3 | 503.63 | 0.318 | 15 | 400 |
| 248.3 | 501.89 | 0.319 | 15 | 400 |
| 318.5 | 413.93 | 0.32 | 41 | 100 |
| 248.3 | 495.97 | 0.322 | 15 | 400 |
| 248.3 | 503.98 | 0.323 | 15 | 400 |
| 248.3 | 504.33 | 0.323 | 15 | 400 |
| 248.3 | 496.32 | 0.323 | 15 | 400 |
| 248.3 | 505.03 | 0.324 | 15 | 400 |
| 248.3 | 499.81 | 0.324 | 15 | 400 |
| 248.3 | 501.2 | 0.325 | 15 | 400 |
| 248.3 | 499.46 | 0.325 | 15 | 400 |
| 248.3 | 505.72 | 0.328 | 15 | 400 |
| 248.3 | 500.85 | 0.329 | 15 | 400 |
| 248.3 | 495.62 | 0.33 | 15 | 400 |
| 248.3 | 496.67 | 0.331 | 15 | 400 |
| 318.5 | 415 | 0.332 | 41 | 100 |
| 248.3 | 501.55 | 0.332 | 15 | 400 |
| 248.3 | 495.27 | 0.343 | 15 | 400 |
| 324.8 | 413.93 | 0.348 | 41 | 200 |
| 318.5 | 413.21 | 0.352 | 41 | 100 |
| 318.5 | 413.57 | 0.36 | 41 | 100 |
| 324.8 | 416.44 | 0.373 | 41 | 200 |
| 318.5 | 412.5 | 0.379 | 41 | 100 |
| 324.8 | 413.57 | 0.398 | 41 | 200 |
| 318.5 | 412.85 | 0.43 | 41 | 100 |
| 324.8 | 416.79 | 0.434 | 41 | 200 |
| 318.5 | 414.65 | 0.452 | 41 | 100 |
| 324.8 | 413.21 | 0.458 | 41 | 200 |
| 324.8 | 417.15 | 0.461 | 41 | 200 |
| 318.5 | 414.29 | 0.482 | 41 | 100 |
| 324.8 | 412.5 | 0.54 | 41 | 200 |
| 318.5 | 411.78 | 0.545 | 41 | 100 |
| 324.8 | 412.85 | 0.552 | 41 | 200 |
| 318.5 | 412.14 | 0.609 | 41 | 100 |
| 318.5 | 410.34 | 0.612 | 41 | 100 |
| 318.5 | 411.42 | 0.617 | 41 | 100 |
| 324.8 | 417.51 | 0.621 | 41 | 200 |
| 318.5 | 410.7 | 0.654 | 41 | 100 |
| 324.8 | 411.06 | 0.687 | 41 | 200 |
| 318.5 | 411.06 | 0.711 | 41 | 100 |
| 324.8 | 417.87 | 0.719 | 41 | 200 |
| 324.8 | 411.42 | 0.761 | 41 | 200 |
| 324.8 | 411.78 | 0.769 | 41 | 200 |
| 324.8 | 422.52 | 0.813 | 41 | 200 |
| 324.8 | 422.88 | 0.816 | 41 | 200 |
| 324.8 | 412.14 | 0.817 | 41 | 200 |
| 324.8 | 418.23 | 0.827 | 41 | 200 |
| 324.8 | 423.23 | 0.852 | 41 | 200 |
| 324.8 | 421.45 | 0.914 | 41 | 200 |
| 324.8 | 422.16 | 0.914 | 41 | 200 |
| 324.8 | 423.95 | 0.917 | 41 | 200 |
| 324.8 | 419.66 | 0.923 | 41 | 200 |
| 324.8 | 418.94 | 0.924 | 41 | 200 |
| 324.8 | 421.8 | 0.926 | 41 | 200 |
| 324.8 | 419.3 | 0.928 | 41 | 200 |
| 324.8 | 420.73 | 0.952 | 41 | 200 |
| 324.8 | 420.37 | 0.955 | 41 | 200 |
| 324.8 | 424.3 | 0.968 | 41 | 200 |
| 324.8 | 418.58 | 0.969 | 41 | 200 |
| 324.8 | 420.02 | 0.972 | 41 | 200 |
| 324.8 | 423.59 | 0.978 | 41 | 200 |
| 324.8 | 412.14 | 1 | 15 | 200 |
| 324.8 | 412.5 | 1 | 15 | 200 |
| 324.8 | 412.85 | 1 | 15 | 200 |
| 324.8 | 413.21 | 1 | 15 | 200 |
| 324.8 | 413.57 | 1 | 15 | 200 |
| 324.8 | 413.93 | 1 | 15 | 200 |
| 324.8 | 415.72 | 1 | 15 | 200 |
| 324.8 | 416.08 | 1 | 15 | 200 |
| 324.8 | 416.44 | 1 | 15 | 200 |
| 324.8 | 416.79 | 1 | 15 | 200 |
| 324.8 | 417.15 | 1 | 15 | 200 |
| 324.8 | 417.51 | 1 | 15 | 200 |
| 324.8 | 428.94 | 1 | 15 | 200 |
| 324.8 | 421.09 | 1.009 | 41 | 200 |
| 324.8 | 428.94 | 1.027 | 41 | 200 |
| 324.8 | 424.66 | 1.032 | 41 | 200 |
| 324.8 | 427.52 | 1.084 | 41 | 200 |
| 324.8 | 425.73 | 1.087 | 41 | 200 |
| 324.8 | 428.59 | 1.104 | 41 | 200 |
| 324.8 | 428.23 | 1.124 | 41 | 200 |
| 324.8 | 425.02 | 1.151 | 41 | 200 |
| 324.8 | 426.09 | 1.155 | 41 | 200 |
| 324.8 | 427.87 | 1.172 | 41 | 200 |
| 324.8 | 427.16 | 1.188 | 41 | 200 |
| 324.8 | 426.45 | 1.194 | 41 | 200 |
| 324.8 | 425.38 | 1.213 | 41 | 200 |
| 318.5 | 415 | 1.224 | 15 | 100 |
| 324.8 | 426.8 | 1.24 | 41 | 200 |
| 318.5 | 414.65 | 1.312 | 15 | 100 |
| 318.5 | 414.29 | 1.49 | 15 | 100 |
| 318.5 | 413.93 | 1.676 | 15 | 100 |
| 318.5 | 413.57 | 1.834 | 15 | 100 |
| 318.5 | 413.21 | 1.916 | 15 | 100 |
| 318.5 | 412.85 | 2.063 | 15 | 100 |
| 318.5 | 412.5 | 2.215 | 15 | 100 |
| 318.5 | 412.14 | 2.669 | 15 | 100 |
| 318.5 | 411.78 | 2.832 | 15 | 100 |
| 318.5 | 411.42 | 3.133 | 15 | 100 |
| 318.5 | 411.06 | 3.482 | 15 | 100 |
| 318.5 | 410.7 | 4.18 | 15 | 100 |
| 318.5 | 410.34 | 4.967 | 15 | 100 |

TABLE 5
Metal identification. Training data table to measure oil Api
(Va = 100; Cu = 200; Ni = 300; Fe = 400).

| Excitation length | Wavelength | Wave Intensity | API | Metal Type |
|---|---|---|---|---|
| 232 | 501.2 | 0.012 | 41 | 300 |
| 232 | 502.24 | 0.013 | 41 | 300 |
| 232 | 503.63 | 0.014 | 41 | 300 |
| 324.8 | 411.78 | 0.017 | 15 | 200 |
| 232 | 502.94 | 0.021 | 41 | 300 |
| 232 | 502.59 | 0.022 | 41 | 300 |
| 232 | 501.89 | 0.023 | 41 | 300 |
| 324.8 | 417.87 | 0.024 | 15 | 200 |
| 232 | 501.55 | 0.025 | 41 | 300 |
| 232 | 503.29 | 0.025 | 41 | 300 |
| 232 | 503.98 | 0.026 | 41 | 300 |
| 232 | 500.85 | 0.027 | 41 | 300 |
| 324.8 | 411.06 | 0.035 | 15 | 200 |
| 324.8 | 415.36 | 0.038 | 15 | 200 |
| 232 | 501.2 | 0.038 | 15 | 300 |
| 232 | 504.33 | 0.038 | 41 | 300 |
| 324.8 | 411.42 | 0.039 | 15 | 200 |
| 232 | 501.55 | 0.039 | 15 | 300 |
| 324.8 | 418.23 | 0.041 | 15 | 200 |
| 232 | 495.62 | 0.042 | 15 | 300 |
| 232 | 502.59 | 0.042 | 15 | 300 |
| 232 | 497.02 | 0.044 | 15 | 300 |
| 232 | 502.24 | 0.044 | 15 | 300 |
| 232 | 500.5 | 0.044 | 41 | 300 |
| 232 | 495.27 | 0.045 | 15 | 300 |
| 232 | 501.89 | 0.046 | 15 | 300 |
| 232 | 497.37 | 0.047 | 15 | 300 |
| 232 | 502.94 | 0.047 | 15 | 300 |
| 232 | 506.76 | 0.047 | 41 | 300 |
| 232 | 500.85 | 0.048 | 15 | 300 |
| 232 | 507.81 | 0.048 | 41 | 300 |
| 232 | 500.15 | 0.049 | 15 | 300 |
| 232 | 506.42 | 0.049 | 41 | 300 |
| 232 | 500.5 | 0.05 | 15 | 300 |
| 232 | 496.67 | 0.051 | 15 | 300 |
| 232 | 505.03 | 0.051 | 41 | 300 |
| 232 | 505.37 | 0.052 | 41 | 300 |
| 232 | 507.11 | 0.052 | 41 | 300 |
| 232 | 507.46 | 0.052 | 41 | 300 |
| 232 | 503.29 | 0.054 | 15 | 300 |
| 232 | 500.15 | 0.054 | 41 | 300 |
| 232 | 499.81 | 0.055 | 15 | 300 |
| 232 | 503.98 | 0.055 | 15 | 300 |
| 232 | 503.63 | 0.056 | 15 | 300 |
| 232 | 497.71 | 0.057 | 15 | 300 |
| 232 | 504.68 | 0.058 | 41 | 300 |
| 232 | 495.97 | 0.059 | 15 | 300 |
| 324.8 | 428.23 | 0.06 | 15 | 200 |
| 232 | 498.06 | 0.06 | 15 | 300 |
| 232 | 504.33 | 0.06 | 15 | 300 |
| 324.8 | 418.58 | 0.061 | 15 | 200 |
| 232 | 496.32 | 0.061 | 15 | 300 |
| 324.8 | 414.65 | 0.062 | 15 | 200 |
| 232 | 499.81 | 0.062 | 41 | 300 |
| 232 | 505.72 | 0.062 | 41 | 300 |
| 232 | 506.42 | 0.063 | 15 | 300 |
| 232 | 497.02 | 0.063 | 41 | 300 |
| 232 | 507.11 | 0.064 | 15 | 300 |
| 232 | 507.46 | 0.064 | 15 | 300 |
| 232 | 506.07 | 0.064 | 41 | 300 |
| 232 | 498.76 | 0.066 | 15 | 300 |
| 232 | 499.11 | 0.068 | 15 | 300 |
| 232 | 506.76 | 0.068 | 15 | 300 |
| 232 | 497.37 | 0.068 | 41 | 300 |
| 232 | 506.07 | 0.07 | 15 | 300 |
| 232 | 507.81 | 0.07 | 15 | 300 |
| 324.8 | 428.59 | 0.073 | 15 | 200 |
| 232 | 504.68 | 0.074 | 15 | 300 |
| 232 | 495.97 | 0.074 | 41 | 300 |
| 232 | 498.41 | 0.075 | 15 | 300 |
| 232 | 496.67 | 0.075 | 41 | 300 |
| 232 | 499.46 | 0.076 | 15 | 300 |
| 232 | 496.32 | 0.076 | 41 | 300 |
| 232 | 497.71 | 0.077 | 41 | 300 |
| 232 | 498.06 | 0.079 | 41 | 300 |
| 324.8 | 418.94 | 0.08 | 15 | 200 |
| 232 | 505.72 | 0.081 | 15 | 300 |
| 232 | 495.62 | 0.081 | 41 | 300 |
| 232 | 499.46 | 0.081 | 41 | 300 |
| 232 | 505.03 | 0.082 | 15 | 300 |
| 324.8 | 414.29 | 0.083 | 15 | 200 |
| 232 | 505.37 | 0.084 | 15 | 300 |
| 232 | 499.11 | 0.085 | 41 | 300 |
| 232 | 495.27 | 0.086 | 41 | 300 |
| 232 | 498.76 | 0.087 | 41 | 300 |
| 324.8 | 415 | 0.096 | 15 | 200 |
| 232 | 498.41 | 0.097 | 41 | 300 |
| 324.8 | 420.02 | 0.099 | 15 | 200 |
| 324.8 | 421.8 | 0.108 | 15 | 200 |
| 324.8 | 419.3 | 0.111 | 15 | 200 |
| 324.8 | 427.87 | 0.116 | 15 | 200 |
| 324.8 | 415.36 | 0.119 | 41 | 200 |
| 324.8 | 421.45 | 0.121 | 15 | 200 |
| 324.8 | 422.16 | 0.121 | 15 | 200 |
| 324.8 | 415.72 | 0.128 | 41 | 200 |
| 324.8 | 422.88 | 0.13 | 15 | 200 |
| 324.8 | 422.52 | 0.143 | 15 | 200 |
| 324.8 | 423.59 | 0.147 | 15 | 200 |
| 324.8 | 419.66 | 0.152 | 15 | 200 |
| 324.8 | 420.73 | 0.165 | 15 | 200 |
| 324.8 | 420.37 | 0.168 | 15 | 200 |
| 324.8 | 427.16 | 0.17 | 15 | 200 |
| 324.8 | 427.52 | 0.172 | 15 | 200 |
| 324.8 | 415 | 0.173 | 41 | 200 |
| 324.8 | 423.23 | 0.18 | 15 | 200 |
| 324.8 | 416.08 | 0.181 | 41 | 200 |
| 324.8 | 421.09 | 0.191 | 15 | 200 |
| 324.8 | 425.02 | 0.211 | 15 | 200 |
| 324.8 | 414.65 | 0.223 | 41 | 200 |
| 324.8 | 426.45 | 0.238 | 15 | 200 |
| 324.8 | 414.29 | 0.247 | 41 | 200 |
| 324.8 | 424.66 | 0.251 | 15 | 200 |
| 324.8 | 426.8 | 0.254 | 15 | 200 |
| 324.8 | 423.95 | 0.261 | 15 | 200 |
| 248.3 | 508.85 | 0.265 | 15 | 400 |
| 248.3 | 509.54 | 0.266 | 15 | 400 |
| 324.8 | 426.09 | 0.267 | 15 | 200 |
| 248.3 | 509.19 | 0.269 | 15 | 400 |
| 248.3 | 509.89 | 0.276 | 15 | 400 |
| 248.3 | 508.5 | 0.277 | 15 | 400 |
| 248.3 | 508.15 | 0.283 | 15 | 400 |
| 248.3 | 507.46 | 0.284 | 15 | 400 |
| 248.3 | 507.81 | 0.287 | 15 | 400 |
| 248.3 | 503.29 | 0.289 | 15 | 400 |
| 248.3 | 506.42 | 0.29 | 15 | 400 |
| 248.3 | 498.06 | 0.293 | 15 | 400 |
| 248.3 | 498.76 | 0.293 | 15 | 400 |
| 248.3 | 497.71 | 0.295 | 15 | 400 |
| 248.3 | 497.02 | 0.302 | 15 | 400 |
| 248.3 | 506.76 | 0.302 | 15 | 400 |
| 248.3 | 499.11 | 0.303 | 15 | 400 |
| 248.3 | 502.94 | 0.303 | 15 | 400 |
| 248.3 | 500.5 | 0.304 | 15 | 400 |
| 324.8 | 424.3 | 0.306 | 15 | 200 |
| 248.3 | 507.11 | 0.306 | 15 | 400 |
| 248.3 | 498.41 | 0.308 | 15 | 400 |
| 248.3 | 505.37 | 0.308 | 15 | 400 |
| 324.8 | 425.73 | 0.31 | 15 | 200 |
| 248.3 | 506.07 | 0.312 | 15 | 400 |
| 324.8 | 425.38 | 0.313 | 15 | 200 |
| 248.3 | 497.37 | 0.314 | 15 | 400 |
| 248.3 | 502.24 | 0.314 | 15 | 400 |
| 248.3 | 500.15 | 0.316 | 15 | 400 |
| 248.3 | 504.68 | 0.317 | 15 | 400 |
| 248.3 | 502.59 | 0.318 | 15 | 400 |
| 248.3 | 503.63 | 0.318 | 15 | 400 |
| 248.3 | 501.89 | 0.319 | 15 | 400 |
| 318.5 | 413.93 | 0.32 | 41 | 100 |
| 248.3 | 495.97 | 0.322 | 15 | 400 |
| 248.3 | 503.98 | 0.323 | 15 | 400 |
| 248.3 | 504.33 | 0.323 | 15 | 400 |
| 248.3 | 505.03 | 0.324 | 15 | 400 |
| 248.3 | 499.81 | 0.324 | 15 | 400 |
| 248.3 | 501.2 | 0.325 | 15 | 400 |
| 248.3 | 499.46 | 0.325 | 15 | 400 |
| 248.3 | 505.72 | 0.328 | 15 | 400 |
| 248.3 | 500.85 | 0.329 | 15 | 400 |
| 248.3 | 495.62 | 0.33 | 15 | 400 |
| 248.3 | 496.67 | 0.331 | 15 | 400 |
| 318.5 | 415 | 0.332 | 41 | 100 |
| 248.3 | 501.55 | 0.332 | 15 | 400 |
| 248.3 | 495.27 | 0.343 | 15 | 400 |
| 324.8 | 413.93 | 0.348 | 41 | 200 |
| 318.5 | 413.21 | 0.352 | 41 | 100 |
| 318.5 | 413.57 | 0.36 | 41 | 100 |
| 324.8 | 416.44 | 0.373 | 41 | 200 |
| 318.5 | 412.5 | 0.379 | 41 | 100 |
| 324.8 | 413.57 | 0.398 | 41 | 200 |
| 318.5 | 412.85 | 0.43 | 41 | 100 |
| 324.8 | 416.79 | 0.434 | 41 | 200 |
| 318.5 | 414.65 | 0.452 | 41 | 100 |
| 324.8 | 413.21 | 0.458 | 41 | 200 |
| 324.8 | 417.15 | 0.461 | 41 | 200 |
| 318.5 | 414.29 | 0.482 | 41 | 100 |
| 324.8 | 412.5 | 0.54 | 41 | 200 |
| 318.5 | 411.78 | 0.545 | 41 | 100 |
| 324.8 | 412.85 | 0.552 | 41 | 200 |
| 318.5 | 412.14 | 0.609 | 41 | 100 |
| 318.5 | 410.34 | 0.612 | 41 | 100 |
| 318.5 | 411.42 | 0.617 | 41 | 100 |
| 324.8 | 417.51 | 0.621 | 41 | 200 |
| 318.5 | 410.7 | 0.654 | 41 | 100 |
| 324.8 | 411.06 | 0.687 | 41 | 200 |
| 318.5 | 411.06 | 0.711 | 41 | 100 |
| 324.8 | 417.87 | 0.719 | 41 | 200 |
| 324.8 | 411.42 | 0.761 | 41 | 200 |
| 324.8 | 411.78 | 0.769 | 41 | 200 |
| 324.8 | 422.52 | 0.813 | 41 | 200 |
| 324.8 | 422.88 | 0.816 | 41 | 200 |
| 324.8 | 412.14 | 0.817 | 41 | 200 |
| 324.8 | 418.23 | 0.827 | 41 | 200 |
| 324.8 | 423.23 | 0.852 | 41 | 200 |
| 324.8 | 421.45 | 0.914 | 41 | 200 |
| 324.8 | 422.16 | 0.914 | 41 | 200 |
| 324.8 | 423.95 | 0.917 | 41 | 200 |
| 324.8 | 419.66 | 0.923 | 41 | 200 |
| 324.8 | 418.94 | 0.924 | 41 | 200 |
| 324.8 | 421.8 | 0.926 | 41 | 200 |
| 324.8 | 419.3 | 0.928 | 41 | 200 |
| 324.8 | 420.73 | 0.952 | 41 | 200 |
| 324.8 | 420.37 | 0.955 | 41 | 200 |
| 324.8 | 424.3 | 0.968 | 41 | 200 |
| 324.8 | 418.58 | 0.969 | 41 | 200 |
| 324.8 | 420.02 | 0.972 | 41 | 200 |
| 324.8 | 423.59 | 0.978 | 41 | 200 |
| 324.8 | 412.14 | 1 | 15 | 200 |
| 324.8 | 412.5 | 1 | 15 | 200 |
| 324.8 | 412.85 | 1 | 15 | 200 |
| 324.8 | 413.21 | 1 | 15 | 200 |
| 324.8 | 413.57 | 1 | 15 | 200 |
| 324.8 | 413.93 | 1 | 15 | 200 |
| 324.8 | 415.72 | 1 | 15 | 200 |
| 324.8 | 416.08 | 1 | 15 | 200 |
| 324.8 | 416.44 | 1 | 15 | 200 |
| 324.8 | 416.79 | 1 | 15 | 200 |
| 324.8 | 417.15 | 1 | 15 | 200 |
| 324.8 | 417.51 | 1 | 15 | 200 |
| 324.8 | 428.94 | 1 | 15 | 200 |
| 324.8 | 421.09 | 1.009 | 41 | 200 |
| 324.8 | 428.94 | 1.027 | 41 | 200 |
| 324.8 | 424.66 | 1.032 | 41 | 200 |
| 324.8 | 427.52 | 1.084 | 41 | 200 |
| 324.8 | 425.73 | 1.087 | 41 | 200 |
| 324.8 | 428.59 | 1.104 | 41 | 200 |
| 324.8 | 428.23 | 1.124 | 41 | 200 |
| 324.8 | 425.02 | 1.151 | 41 | 200 |
| 324.8 | 426.09 | 1.155 | 41 | 200 |
| 324.8 | 427.87 | 1.172 | 41 | 200 |
| 324.8 | 427.16 | 1.188 | 41 | 200 |
| 324.8 | 426.45 | 1.194 | 41 | 200 |
| 324.8 | 425.38 | 1.213 | 41 | 200 |
| 318.5 | 415 | 1.224 | 15 | 100 |
| 324.8 | 426.8 | 1.24 | 41 | 200 |
| 318.5 | 414.65 | 1.312 | 15 | 100 |
| 318.5 | 414.29 | 1.49 | 15 | 100 |
| 318.5 | 413.93 | 1.676 | 15 | 100 |
| 318.5 | 413.57 | 1.834 | 15 | 100 |
| 318.5 | 413.21 | 1.916 | 15 | 100 |
| 318.5 | 412.85 | 2.063 | 15 | 100 |
| 318.5 | 412.5 | 2.215 | 15 | 100 |
| 318.5 | 412.14 | 2.669 | 15 | 100 |
| 318.5 | 411.78 | 2.832 | 15 | 100 |
| 318.5 | 411.42 | 3.133 | 15 | 100 |
| 318.5 | 411.06 | 3.482 | 15 | 100 |
| 318.5 | 410.7 | 4.18 | 15 | 100 |
| 318.5 | 410.34 | 4.967 | 15 | 100 |

TABLE 6
Correlation between type of petroleum oil (API gravity)
and emission according the light expression
Type of petroleum 0-15 API Gravity

| | |
|---|---|
| Concentration 10x | Concentration 1x |

| Emission | Intensity | Emission | Intensity |
|---|---|---|---|
| Wavelength | (mean +/− SD) | Wavelength | (mean +/− SD) |
| 498.95 | 18.35 | 341.19 | 3.04 |
| 499.31 | 18.69 | 342.34 | 5 |
| 517.47 | 20.85 | 498.95 | 3 |
| 517.83 | 20.74 | 499.31 | 3 |
| 518.20 | 22.91 | 518.2 | 2 |
| 518.56 | 19.74 | 544.49 | 2 |
| 544.49 | 21.83 | 544.85 | 4 |
| 544.85 | 24.44 | 1019.81 | 2.16 |

*Wavelength excitation 335

TABLE 7
Type of petroleum 0-15 API Gravity

| | |
|---|---|
| Concentration 10x | Concentration 1x |

| Emission | Intensity | Emission | Intensity |
|---|---|---|---|
| Wavelength | (mean +/− SD) | Wavelength | (mean +/− SD) |
| 499.31 | 17.19621622 | 342.34 | 6 |
| 517.47 | 20.27027027 | 516.39 | 2.85 |
| 517.83 | 20.09432432 | 518.2 | 4 |
| 518.2 | 22.12324324 | 544.49 | 4 |
| 518.56 | 18.83945946 | 544.85 | 6 |
| 538.39 | 17.15783784 | 1019.54 | 4.17 |
| 544.49 | 21.43648649 | 1019.81 | 6 |
| 544.85 | 23.88567568 | 1020.36 | 4.75 |

*Wavelength excitation 355

TABLE 8
Type of petroleum 0-15 API Gravity

| | |
|---|---|
| Concentration 10x | Concentration 1x |

| Emission | Intensity | Emission | Intensity |
|---|---|---|---|
| Wavelength | (mean +/− SD) | Wavelength | (mean +/− SD) |
| 517.47 | 13.54513514 | 341.96 | 7.606666667 |
| 517.83 | 13.54783784 | 342.34 | 8.051818182 |
| 518.2 | 15.54621622 | 498.95 | 7.339459459 |
| 518.56 | 12.29459459 | 499.31 | 7.292432432 |
| 538.39 | 12.25243243 | 518.2 | 8.739189189 |
| 539.11 | 12.04594595 | 544.49 | 8.971621622 |
| 544.49 | 16.05189189 | 544.85 | 11.66324324 |
| 544.85 | 18.68 | 1019.81 | 9.413 |

*Wavelength excitation 375

TABLE 9
Type of petroleum 0-15 API Gravity

| | |
|---|---|
| Concentration 10x | Concentration 1x |

| Emission | Intensity | Emission | Intensity |
|---|---|---|---|
| Wavelength | (mean +/− SD) | Wavelength | (mean +/− SD) |
| 450.08 | 207.9064865 | 449.71 | 342.81 |
| 450.45 | 216.1283784 | 450.08 | 350.4113514 |
| 450.82 | 214.7264865 | 450.45 | 359.9508108 |
| 451.19 | 213.3221622 | 450.82 | 359.0281081 |
| 451.56 | 215.2410811 | 451.19 | 357.3110811 |
| 451.93 | 209.9402703 | 451.56 | 357.8843243 |
| 452.3 | 209.3167568 | 451.93 | 350.7759459 |
| 452.67 | 206.5167568 | 452.3 | 347.8286486 |

*Wavelength excitation 450
*Different dilution samples were used. Dilution factor 10.
*Cyclohexane was used to dilute the petroleum samples.
*Ocean optic USB2000+ was used as the optical sensor to measure the petroleum fluorescence.
Measurements were taken every 5 seconds during 3 minutes
*Fluorescence intensity was determined based on excitation wavelength.

1. A method for determining the presence of petroleum in a sample, the method comprising:
a. obtaining a sample that may or may not contain petroleum; and
b. detecting the presence of fluorescence produced by the sample, wherein the presence of fluorescence indicates the presence of petroleum in the sample.

2. A method for determining the grade of petroleum in a sample, the method comprising:
a. obtaining a sample comprising petroleum;
b. quantifying the amount of fluorescence produced by the petroleum in the sample;
c. comparing the amount of fluorescence in the sample to a standard curve, where the curve correlates the amount of fluorescence to the grade of petroleum; and
d. identifying the grade of petroleum in the sample.

3. The method of claim 1 embodied in a computer program product for execution on an instruction processing system, comprising a tangible storage medium readable by the instruction processing system and storing instructions for execution by the instruction processing system for performing the method comprising:
a. obtaining a sample that may or may not contain petroleum; and
b. detecting the presence of fluorescence produced by the sample, wherein the presence of fluorescence indicates the presence of petroleum in the sample.

4. The method of claim 2 embodied in a computer program product for execution on an instruction processing system, comprising a tangible storage medium readable by the instruction processing system and storing instructions for execution by the instruction processing system for performing the method comprising:
a. obtaining a sample comprising petroleum;
b. quantifying the amount of fluorescence produced by the petroleum in the sample;
c. comparing the amount of fluorescence in the sample to a standard curve, where the curve correlates the amount of fluorescence to the grade of petroleum; and
d. identifying the grade of petroleum in the sample.

5. The method of claim 1, wherein the sample comprises a soil sample.

6. The method of claim 5, wherein the sample comprises sandy soil, sludge, clay sediment, sandy sediment, or any combination thereof.

7. The method of claim 1, wherein the sample comprises of petroleum comprises vanadium, nickel, iron, copper, or any combination thereof.

8. The method of claim 1, wherein step (b) comprises (1) exposing the sample to UV light and (2) detecting and/or quantifying the amount of fluorescence by an optical fiber sensor.

9. A computer program product for determining the grade of petroleum in a sample, the computer program product comprising:
a tangible storage medium readable by a computer system and storing instructions for execution by the computer system for performing a method comprising:
a. determining a calibration curve that correlates the amount of fluorescence to the grade of petroleum the sample;
b. determining the amount of fluorescence produced by the petroleum in the sample;
c. comparing the amount of fluorescence produced by the petroleum in the sample to the calibration curve; and
d. calculating the grade of petroleum in a sample.

10. A system for determining the grade of petroleum in a sample on an instruction processing system, comprising:
a tangible storage medium readable by the instruction processing system and storing instructions for execution by the instruction processing system;
a calibration curve that correlates the amount of fluorescence to the grade of petroleum the sample;
a quantification module for obtaining the amount of fluorescence produced by the petroleum in the sample;
a comparison module for comparing the amount of fluorescence produced by the petroleum in the sample to the calibration curve; and
a calculating module for determining the grade of petroleum in a sample.

11. The method of claim 2, wherein the sample comprises a soil sample.

12. The method of claim 11, wherein the sample comprises sandy soil, sludge, clay sediment, sandy sediment, or any combination thereof.

13. The method of claim 2, wherein the sample comprises of petroleum comprises vanadium, nickel, iron, copper, or any combination thereof.

14. The method of claim 2, wherein step (b) comprises (1) exposing the sample to UV light and (2) detecting and/or quantifying the amount of fluorescence by an optical fiber sensor.
